(19) United States
(12) Patent Application Publication
(10) Pub. No.: US 2024/0238341 A1
(43) Pub. Date: Jul. 18, 2024

(54) NEUROPROTECTIVE COMPOSITIONS AND METHODS

(71) Applicant: Washington University, St. Louis, MO (US)

(72) Inventor: Jonathan Kipnis, St. Louis, MO (US)

(21) Appl. No.: 18/561,846

(22) PCT Filed: May 20, 2022
(86) PCT No.: PCT/US2022/030332

§ 371 Date: Nov. 17, 2023

Related U.S. Application Data
(60) Provisional application No. 63/191,290, filed on May 20, 2021.

(51) Int. Cl. A61K 35/17; A61K 39/00; A61P 25/00; C12N 5/0783; C12Q 1/6869; G01N 33/569; G01N 33/68

(52) U.S. Cl. CPC A61K 35/17; A61K 39/4611; A61K 39/4632; A61K 39/46433; A61P 25/00; C12N 5/0636; C12Q 1/6869; G01N 33/56972; G01N 33/6848; G01N 2333/7051; G01N 2333/70539

(57) ABSTRACT

The present disclosure is directed to methods and compositions for producing a recombinant cell expressing a T cell receptor (TCR) specific for a peptide of interest, methods and compositions for obtaining a nucleic acid or pair of TCR chain polypeptides and/or nucleic acids encoding a TCR, a cell population comprising the recombinant cell harboring the one or more nucleic acids encoding a TCR or TCR chain obtained by said method, and a method for treating a disorder of the central nervous system comprising administering to the subject said cell population.

### CROSS REFERENCE TO RELATED APPLICATIONS

This application claims the benefit of U.S. Provisional Application No. 63/191,290, filed May 20, 2021 the disclosure of which is hereby incorporated by reference in its entirety.

### FIELD OF THE TECHNOLOGY

The present disclosure relates to personalized cell-based therapeutics in the treatment of central nervous system disorders.

### REFERENCE TO SEQUENCE LISTING

This application contains a Sequence Listing that has been submitted in ASCII format via EFS-Web and is hereby incorporated by reference in its entirety. The ASCII copy, created on May 20, 2022, is named Untitled_ST25.txt, and is 12,288 bytes in size.

### BACKGROUND

Traumatic central nervous system injuries currently have limited effective treatments, especially when the injury is severe. Palliative methods that reduce swelling or coma-inducing medication that give the brain time to heal are often undertaken, but they do not address the underlying tissue damage. Finally, surgery is employed to remove debris or necrotic tissues in order to minimize additional damage to the brain.

Neurological disorders can also be treated with drugs and biologics, but both approaches are dose limited in their ability to cross the blood brain barrier. Emerging therapies include mesenchymal stem cell, dendritic cells, and exosomes, but their efficacies have not been conclusively demonstrated.

T cells have been shown to protect neurons from degeneration, but to date safe and effective therapies using T cells to impart neuroprotection to injured CNS tissues in human subjects have yet to be developed.

There is a need for compositions and methods for treating and preventing neuronal damage resulting from traumatic central nervous system injuries, stroke and neurodegeneration.

### SUMMARY

The present disclosure provides, in part, methods for personalized cell-based therapeutics in the treatment of central nervous system disorders.

In one aspect, the present disclosure provides methods of treating or preventing a central nervous system (CNS) injury in a subject in need thereof, the methods generally comprise a) obtaining a biological sample from the subject at a CNS injury site wherein the sample comprises infiltrating lymphocytes; b) isolating the infiltrating lymphocytes, and sequencing a T cell receptor (TCR) or part thereof, expressed by the lymphocytes; c) isolating a MHC-peptide complex from the biological sample and identifying a pool of self-peptides; d) expressing a T cell receptor identified in step b) in an immune effector cell; e) screening a TCR-expressing immune effector cell generated in step d) against the self-peptide identified in step c) and selecting TCR-expressing immune effector cells with affinity to the self-peptide; and f) administering a population of TCR-expressing immune effector cells selected in step e) to the subject, thereby treating or preventing the CNS injury.

In another aspect, the present disclosure provides methods of treating or preventing neuronal death from a stroke a subject in need thereof, the methods generally comprise a) obtaining a biological sample from the subject, wherein the sample comprises infiltrating lymphocytes; b) isolating the infiltrating lymphocytes, and sequencing a T cell receptor (TCR) or part thereof expressed by the lymphocytes; c) isolating a MHC-peptide complex from the biological sample identifying a pool of self-peptides; d) expressing a T cell receptor identified in step b) in an immune effector cell; e) screening a TCR-expressing immune effector cell generated in step d) against the self-peptide identified in step c) and selecting TCR-expressing immune effector cells with affinity to the self-peptide; and f) administering a population of TCR-expressing immune effector cells selected in step e) to the subject, thereby treating or preventing the CNS injury.

In still another aspect of the present disclosure provides methods of treating or preventing neurodegeneration in a subject in need thereof, the methods generally comprise a) obtaining a biological sample from the subject at a site of neurodegeneration, wherein the sample comprises infiltrating lymphocytes; b) isolating the infiltrating lymphocytes, and sequencing a T cell receptor (TCR) or part thereof expressed by the lymphocytes; c) isolating a MHC-peptide complex from the biological sample identifying a pool of self-peptides; d) expressing a T cell receptor identified in step b) in an immune effector cell; e) screening a TCR-expressing immune effector cell generated in step d) against the self-peptide identified in step c) and selecting TCR-expressing immune effector cells with affinity to the self-peptide; and f) administering a population of TCR-expressing immune effector cells selected in step e) to the subject, thereby treating or preventing the CNS injury.

In each of the above aspects, the biological sample can be previously obtained from the subject. In each of the above aspects, the biological sample can be a tissue biopsy or cerebral spinal fluid. In each of the above aspects, the infiltrating lymphocytes comprise T cells. In some embodiments, the T cells are CD4⁺ T cells or CD8⁺ T cells. In each of the above aspects, single-cell RNA sequencing is performed for single-cell assessment of cellular gene expression of the lymphocytes in step b). In some embodiments, the sequencing includes V(D)J sequencing. In some embodiments, the cellular gene expression of the lymphocytes is compared to a reference gene expression from a T cell or population of T cells with naïve T cell features. In some embodiments, lymphocytes are grouped by CDR3 region from the V(D)J sequencing and cells sharing the same sequence of TCRα and TCRβ pair are grouped as clones.

In each of the above aspects, Mass Spec is used to identify the pool of self-peptides. In some embodiments, the MHC is an MHC-II-peptide complex.

In each of the above aspects, the identified TCR can be stably expressed in the immune effector cell thereby generating the TCR-expressing immune effector cell. In each of the above aspects, the identified TCR can be expressed using mRNA thereby generating the TCR-expressing immune effector cell transiently.

In some embodiments, the immune effector cell of step d) is a T cell. In some embodiments, T cell is a CD4⁺ T cell. In some embodiments, screening in step e) includes co-culturing TCR-expressing immune effector cells with a self-peptide identified and step c) and measuring proliferation and/or cytokine secretion. In some embodiments, ELISA or ELISpot is used. In some embodiments, the TCR-expressing immune effector cells which are activated by the self-peptides identified in step c) are selected for administration to the subject. In some embodiments, the cells are graded from low to high activation. In some embodiments, the cells graded with low activation are selected for administration to the subject. In some embodiments, the injured CNS tissue comprises an injured spinal cord, an injured brain, an injured retina, and any combination thereof. In some embodiments, the CNS injury is associated with at least one of CNS trauma, autoimmunity, infection, aging, and chronic neurodegeneration.

### BRIEF DESCRIPTION OF THE FIGURES

The patent or patent application file contains at least one photograph executed in color. Copies of this patent or patent application publication with color photographs will be provided by the Office upon request and payment of the necessary fee.

FIG. 1A-FIG. 1P show clonality of auto-immune T cells in injury site after spinal cord injury. FIG. 1A shows immunohistochemistry of injury site of mouse spinal cord and CD3⁺ T cells 14 days after injury. Insets represent enlarged regions of particular area of interest. FIG. 1B shows a schematic representation of T cell isolation and single cell sequencing. For injured spinal cord sample, n=30 total mice were pooled for T cell isolation. For T cell sorting, CD45⁺ Thy1.2⁺ CD4⁺/CD8⁺ T cells were sorted by flow cytometry. FIG. 1C, FIG. 1D, and FIG. 1E show UMAP visualization of scRNA-seq of conventional T cell from injured spinal cord or homeostatic blood based on gene expression or sample. FIG. 1F shows violin plots showing the expression levels of Cd4 and Cd8a in group CD4 and CD8. FIG. 1G shows UMAP visualization of T cell clonality. Red dots represent T cells with clonality. FIG. 1H shows pie graph showing clonality of CD4⁺ T cell and CD8⁺ T cell in each injured spinal cord sample and homeostatic blood sample. FIG. 1I and FIG. 1J shows Q-plot visualization of each clonally expended T cells. Cells with shared CDR3 region of TCR α chain and β chain pair are shown. CD4⁺ T cells are highlighted. FIG. 1K shows schematic representation of NFAT-GFP reporter system and TCR reconstitution. FIG. 1L and FIG. 1M show flow cytometry analysis and quantification of GFP signal in specific TCR expressing reporter hybridoma after co-culturing with different peptides. FIG. 1N shows a schematic representation of identification of MHC-II binding peptides. FIG. 1O shows CNS elevated protein name in injured spinal cord sample and spinal cord meninges are shown by analyzing expression specificity of peptide targets from Mass spectrometry. FIG. 1P shows ELISA result of IL-2 secretion from co-culturing Cp hybridoma with different peptide candidates.

FIG. 2A-FIG. 2O show transient artificially-autoimmune T cells (taaT cells) improve recovery after CNS injury. FIG. 2A shows a schematic representation of TCR reconstitution in primary CD4⁺ T cells by retro-virus infection. FIG. 2B shows T cell proliferation assay to test function of reconstituted TCR. FIG. 2C shows whole-mount immunohistochemistry of retinal ganglion cells with/out optic nerve injury by Brn-3a staining. Enlarged regions with RGCs were shown in insets. FIG. 2E shows quantification of retinal ganglion cells. Screening function of all the CD4⁺ T cell clone TCRs using optic nerve injury model. *p<0.05, **p<0.01 (one-way ANOVA). FIG. 2F shows quantification of retinal ganglion cells. Testing the function of a diaphragm T cell clone TCR D1 by optic nerve injury model *p<0.05 (one-way ANOVA). FIG. 2D shows schematic representation of optic nerve injury model. FIG. 2G shows quantification of fluoro-gold positive retinal ganglion cells. *p<0.05, **p<0.01, ***p<0.001 (one-way ANOVA). FIG. 2H shows schematic representation of spinal cord injury model to test function of T cell therapy. FIG. 2I shows the Basso Mouse Scale (BMS) score of spinal cord injury mice given different TCR-T treatment. Asterisks indicate a significant difference between PBS control group and the TCR-Cp group (two-way ANOVA with Turkey's post hoc test). FIG. 2J shows schematic representation of transient TCR reconstitution in primary CD4⁺ T cells by mRNA electroporation. FIG. 2K shows T cell proliferation assay to test function of transient reconstituted TCR. FIG. 2L shows quantification of retinal ganglion cells. Testing the function of mRNA-TCR therapy by optic nerve injury. FIG. 2M shows BMS score of spinal cord injury mice given mRNA-TCR therapy. Asterisks indicate a significant difference between control group and the mRNA-Cp/Du group (two-way ANOVA with Turkey's post hoc test). FIG. 2N and FIG. 2O show immunohistochemistry and quantification of scar region of mouse spinal cord after mRNA-TCR treatment.

FIG. 3A-FIG. 3M show therapeutic T cells tune local immune response after spinal cord injury. FIG. 3A shows immunohistochemistry of injury site of mouse spinal cord and myeloid cells 7 days after injury. Insets represent enlarged regions of particular area of interest. FIG. 3B shows UMAP visualization of scRNA-seq of CD45⁺ population from ±5 mm from injury site of spinal cord based on cell types. n=4 total pooled mice per sample. FIG. 3C shows a graph showing cluster proportions in each group. FIG. 3D shows UMAP visualization of three sub-clusters of microglia. FIG. 3E Graph showing sub-cluster proportions of microglia in each group. FIG. 3F shows ten selected upregulated Gene Ontology terms comparing TCR-Cp group with PBS control in microglia. FIG. 3G shows eight selected downregulated Gene Ontology terms comparing TCR-Cp group with PBS control in microglia. FIG. 3H shows a dot plot demonstrating scaled marker gene expression and percentage of cells expressing these genes in each sub-cluster of microglia. FIG. 3I shows UMAP visualization of six sub-clusters of macrophages in the injury site. FIG. 3J shows a dot plot demonstrating scaled marker gene expression and percentage of cells expressing these genes in each sub-cluster of macrophages. FIG. 3K shows a graph showing sub-cluster proportions of macrophages in each group. FIG. 3L shows ten selected upregulated Gene Ontology terms comparing TCR-Cp group with PBS control in macrophages. FIG. 3M shows ten selected upregulated Gene Ontology terms comparing TCR-Cp group with PBS control in dendritic cells.

FIG. 4A shows a schematic representation of using optic nerve injury to test neuron-protective function of T cells. FIG. 4B shows flow cytometry analysis of T cell before injection. FIG. 4C shows quantification of retinal ganglion cells. Testing the function of T cells from mice with different TCR expression by optic nerve injury model *p<0.05 (one-way ANOVA). FIG. 4D and FIG. 4E show quantification of flow cytometry data of T cell infiltration in injured optic nerve. FIG. 4F shows a list of sequences of TCR CDR3 region of all the CD4⁺ T cell clones (SEQ ID NOs: 1-14). FIG. 4G shows sequences of peptides using for NFAT-GFP reporter cell co-culturing (SEQ ID NOs 15-20). FIG. 4H shows quantification of GFP signal and ELISA results of IL-2 secretion in specific TCR expressing reporter hybridoma after co-culturing with different peptides.

FIG. 5A shows a dot plot demonstrating scaled marker gene expression and percentage of cells expressing these genes in each cluster of T cells.

FIG. 5B shows UMAP representation of the scaled average expression of marker genes to identify T cell subtype. FIG. 5C and FIG. 5D shows Qplot representation of the enrichment of Vα and Vβ subtype in CD4⁺ T cells and CD8⁺ T cells comparing with TCRs from injured spinal cord and homeostatic blood.

FIG. 6A shows flow cytometry analysis of primary CD4⁺ T cell expressing each TCR to identify T cell subtype by detecting transcription factor of each CD4⁺ T cell subtype. Th1 (T-bet), Th2 (Gata3), Th17 (RORyt), Treg (Foxp3). FIG. 6B shows schematic representation of Fluoro-gold injection and optic nerve injury model. FIG. 6C shows flow cytometry analysis of activation level of 2D2tg T cell before injection. FIG. 6D shows quantification of retinal ganglion cells. Testing the function of 2D2tg T cells with different activation level by optic nerve injury model *p<0.05 (one-way ANOVA). FIG. 6E shows quantification of flow cytometry data for T cell infiltration in injured optic nerve. FIG. 6F shows schematic representation of spinal cord injury to test function of T cell (one dose) expressing different TCRs. FIG. 6G shows BMS score of spinal cord injury mice given T cell expressing different TCRs. Asterisks indicate a significant difference between PBS control group and TCR-Cp group (two-way ANOVA with Turkey's post hoc test). FIG. 6H shows BMS score of spinal cord injury mice given T cells with/out 2D2 TCR expression. There is no significant difference between the two groups. FIG. 6I shows flow cytometry analysis of 2D2 CD4⁺ T cell infiltration in injury site and non-injury site of spinal cord one month after spinal cord injury. FIG. 6J shows tissue staining of mRNA-GFP and mRNA-Cp.

FIG. 7A shows a heatmap demonstrating expression of marker genes in each cell cluster. FIG. 7B and FIG. 7C show volcano plots with genes that were significantly upregulated or downregulated in microglia (b) and microphages (c) comparing with TCR-Cp treatment with PBS control. FIG. 7D shows violin plots showing the expression levels of Cd74 and Isg15, Ifitm3 and Dab2 of each cell in each group. FIG. 7E and FIG. 7F show ten selected upregulated Gene Ontology terms comparing TCR-Cp treatment group with diaphragm TCR-D1 group in microglia and macrophages. FIG. 7G and FIG. 7H show seven selected downregulated Gene Ontology terms comparing TCR-Cp treatment group with diaphragm TCR-D1 in microglia and macrophages. FIG. 7I shows a schematic representation of FACS-seq of TCR-Cp T cells in injured spinal cord with TCR-Cp cells in spleen as control. n=6 total mice were pooled for T cell isolation FIG. 7J shows UMAP visualization of FACS-seq of TCR-Cp T cells from injured spinal cord and spleen. Dot plot demonstrating scaled marker gene expression and percentage of cells expressing these genes. FIG. 7K shows volcano plots with genes that were significantly upregulated or downregulated in TCR-Cp T cell from injured spinal cord and spleen. FIG. 7L shows top ten upregulated Gene Ontology terms comparing TCR-Cp T cells from injured spinal cord with that in spleen.

FIG. 8 is a schematic showing the concept of the disclosure.

FIG. 9A is a schematic showing the isolation and sequencing of the infiltrating lymphocytes. FIG. 9B is a schematic showing TCR reconstitution. FIG. 9C is a schematic showing the confirmation of function of TCR clones. FIG. 9D is a schematic mechanism. FIG. 9E is a schematic showing the therapy development.

FIG. 10A shows the nucleic acid sequence for TCRα (SEQ ID NO: 21) and TCRb (SEQ ID NO: 22) of the Cp clone. FIG. 10B shows the nucleic acid sequence for TCRα (SEQ ID NO: 23) and TCRb (SEQ ID NO: 24) of the Du clone. FIG. 10C shows the nucleic acid sequence for TCRα (SEQ ID NO: 25) and TCRb (SEQ ID NO: 26) of the Eo clone. FIG. 10D shows the nucleic acid sequence for TCRα (SEQ ID NO: 27) and TCRb (SEQ ID NO: 28) of the Ep clone. FIG. 10E shows the nucleic acid sequence for TCRα (SEQ ID NO: 29) and TCRb (SEQ ID NO: 30) of the Fl clone. FIG. 10F shows the nucleic acid sequence for TCRα (SEQ ID NO: 31) and TCRb (SEQ ID NO: 32) of the Go clone.

### DETAILED DESCRIPTION

The present disclosure is based, at least in part, on the discovery of brain antigen-specific T cells which target to injury sites in the central nervous system and aid neuroprotection and repair. T cell receptors (TCR) derived from the brain antigen-specific T cells, as described herein, are useful to genetically modify an immune effector cell (e.g. CD4⁺ T cell or CD8⁺ T cell) for use in adoptive cell therapy which allows an effective treatment traumatic brain injury, stroke, and neurodegeneration. In particular, the present disclosure provides methods for identifying and isolating TCRs or functional parts thereof affinity against brain-associated antigens presented on MHC molecule (e.g. MOG, MBP, and Licam) from subjects suffering from a CNS disorder. Genetic modification of immune effector cells which express the TCR or functional parts to tune the local immune response. For example, coordinate the recruitment of other immune cells that help protect and repair tissue damage associated with a CNS injury when administered to the subject. Thus, the present disclosure provides treatment methods which include identification and administration of subject specific TCR-expressing immune effector cells.

In various aspects, the disclosure provides methods of treating a subject having a CNS injury, stroke or neurodegeneration that includes administering modified immune effector cells thereby protecting and treating neuron damage. In some aspects, the modified immune effector cells are specific for certain brain antigens of the subject and the subject's particular pathology, and thus when administered to the subject facilitate migration and accumulation to the injury site in the CNS. In certain embodiments, the modified immune effector cells help drive restorative immune responses involving lymphoid and myeloid (CD11 b⁺) cells. In some aspects, the TCR of the modified T cells may be further modified, and the cells expanded ex vivo akin to the CAR-T technology to select the safest and most effective clones for cell therapy.

As described in the examples below, T cells were shown to react after CNS injury in mice. The T cells from injured spinal cords of mice were isolated and the T cell receptors (TCRs) were sequenced. The most prevalent TCRs were cloned and expressed on T cell bodies in a manner similar to CAR T cell technology. Compositions that included T cells modified to express the TCRs from the injured spinal cord tissues were injected into injured mice. The most prevalent clones demonstrated neuroprotection that resulted in improved neuronal survival in the injured mice.

### I. Definitions

The term “a” or “an” entity refers to one or more of that entity; for example, a “polypeptide subunit” is understood to represent one or more polypeptide subunits. As such, the terms “a” (or “an”), “one or more,” and “at least one” can be used interchangeably herein.

Furthermore, “and/or” where used herein is to be taken as specific disclosure of each of the specified features or components with or without the other. Thus, the term “and/or” as used in a phrase such as “A and/or B” herein is intended to include “A and B,” “A or B,” “A” (alone), and “B” (alone).

Unless defined otherwise, technical and scientific terms used herein have the same meaning as commonly understood by one of ordinary skill in the art to which this disclosure is related.

Where applicable, units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. Nucleic acid sequences are written from 5′ to 3′, left to right.

The headings provided herein are not limitations of the various aspects and embodiments of the disclosure, which can be had by reference to the specification as a whole.

Terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of amino acid monomers linearly linked by peptide bonds (also known as amide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-standard amino acids. A polypeptide can be derived from a natural biological source or produced by recombinant technology but is not necessarily translated from a designated nucleic acid sequence. It can be generated in any manner, including by chemical synthesis.

A “protein” as used herein can refer to a single polypeptide, i.e., a single amino acid chain as defined above, but can also refer to two or more polypeptides that are associated, e.g., by disulfide bonds, hydrogen bonds, hydrophobic interactions, etc., to produce, e.g., a multimeric protein.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” when referring to polypeptide subunit or multimeric protein as disclosed herein can include any polypeptide or protein that retain at least some of the activities of the complete polypeptide or protein, but which is structurally different. Fragments of polypeptides include, for example, proteolytic fragments, as well as deletion fragments. Variants include fragments as described above, and also polypeptides with altered amino acid sequences due to amino acid substitutions, deletions, or insertions. Variants can occur spontaneously or be intentionally constructed. Intentionally constructed variants can be produced using art-known mutagenesis techniques. Variant polypeptides can comprise conservative or non-conservative amino acid substitutions, insertions, and/or deletions. Derivatives are polypeptides that have been altered so as to exhibit additional features not found on the native polypeptide, such as increased resistance to proteolytic degradation. Examples include fusion proteins. Variant polypeptides can also be referred to herein as “polypeptide analogs.” As used herein a “derivative” also refers to a subject polypeptide having one or more amino acids chemically derivatized by reaction of a functional side group. Also included as “derivatives” are those peptides that contain one or more standard or synthetic amino acid derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate protein activity are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:.412-417 (1997)).

As used herein, the term “TCR” has its general meaning in the art and refers to the molecule found on the surface of T cells that is responsible for recognizing antigens bound to MHC molecules. During antigen processing, antigens are degraded inside cells and then carried to the cell surface in the form of peptides bound to major histocompatibility complex (MHC) molecules (human leukocyte antigen or HLA molecules in humans). T cells are able to recognize these peptide-MHC complex at the surface of professional antigen presenting cells or target tissue cells. There are two different classes of MHC molecules: MHC Class I and MHC Class II that deliver peptides from different cellular compartments to the cell surface that are recognized by CD8⁺ and CD4⁺ T cells, respectively. The T cell receptor or TCR is the molecule found on the surface of T cells that is responsible for recognizing antigens bound to MHC molecules. The TCR heterodimer consists of an alpha and beta chain in 95% of T cells, whereas 5% of T cells have TCRs consisting of gamma and delta chains. Engagement of the TCR with antigen and MHC results in activation of its T lymphocyte through a series of biochemical events mediated by associated enzymes, co-receptors, and specialized accessory molecules. Each chain of the TCR is a member of the immunoglobulin superfamily and possesses one N-terminal immunoglobulin (Ig)-variable (V) domain, one Ig-constant (C) domain, a transmembrane region, and a short cytoplasmic tail at the C-terminal end. The constant domain of the TCR consists of short connecting sequences in which a cysteine residue forms a disulfide bond, making a link between the two chains. The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3 chains, together with the TCR, form what is known as the TCR complex. The signal from the TCR complex is enhanced by simultaneous binding of the MHC molecules by a specific co-receptor. On helper T cells, this co-receptor is CD4 (specific for class II MHC); whereas on cytotoxic T cells, this co-receptor is CD8 (specific for class I MHC). The co-receptor not only ensures the specificity of the TCR for an antigen, but also allows prolonged engagement between the antigen presenting cell and the T cell and recruits essential molecules (e.g., LCK) inside the cell involved in the signaling of the activated T lymphocyte. The term “T-cell receptor” is thus used in the conventional sense to mean a molecule capable of recognizing a peptide when presented by an MHC molecule. The molecule may be a heterodimer of two chains α and ρ (or optionally γ and δ) or it may be a recombinant single chain TCR construct. The variable domain of both the TCR α-chain and β-chain have three hypervariable or complementarity determining regions (CDRs). CDR3 is the main CDR responsible for recognizing processed antigen. Its hypervariability is determined by recombination events that bring together segments from different gene loci carrying several possible alleles. The genes involved are V and J for the TCR α-chain and V, D and J for the TCR β-chain. Further amplifying the diversity of this CDR3 domain, random nucleotide deletions and additions during recombination take place at the junction of V-J for TCR α-chain, thus giving rise to V(N)J sequences; and V-D and D-J for TCR β-chain, thus giving rise to V(N)D(N)J sequences. Thus, the number of possible CDR3 sequences generated is immense and accounts for the wide capability of the whole TCR repertoire to recognize a number of disparate antigens. At the same time, this CDR3 sequence constitutes a specific molecular fingerprint for its corresponding T cell.

By “specifically binds,” it is meant that a binding molecule, e.g., a TCR or antigen-binding fragment thereof binds to an epitope via its antigen binding domain, and that the binding entails some recognition between the antigen binding domain and the epitope. According to this definition, a TCR is said to “specifically bind” to an epitope when it binds to that epitope, via its antigen-binding domain binds more readily than it would bind to a random, unrelated epitope.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition, or disorder or those in which the disease, condition or disorder is to be prevented.

As used herein, the term “preventing” or “prevention” or “prophylactic treatment” refers to a reduction in risk of acquiring or developing a disease or disorder (i.e., causing at least one of the clinical symptoms of the disease not to develop) in a subject. The subject may or may not be predisposed to the disease in advance of disease onset. As used herein, the term “prophylaxis” is related to “prevention,” and refers to a measure to prevent, rather than to treat or cure a disease.

The term “pharmaceutical composition” refers to a preparation that is in such form as to permit the biological activity of the active ingredient to be effective and does not contain components that are unacceptably toxic to a subject to which the composition would be administered. Such composition can be sterile.

An “effective amount” as disclosed herein is an amount sufficient to carry out a specifically stated purpose. An “effective amount” can be determined empirically and in a routine manner, in relation to the stated purpose.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

As used herein, an “immune effector cell” is a leukocyte that can modulate an immune response. Immune effector cells include T cells, B cells, natural killer cells, invariant T cell receptor alpha natural killer T cells and macrophages. T cell receptor-expressing immune effector cells include T cells which have been engineered to express a T cell receptor. Immune effector cells may be obtained or derived/generated from any appropriate source, such as including, but not limited to, healthy donors, peripheral blood mononuclear cells, cord blood, and induced pluripotent stem cells.

### II. Methods

Applicant has discovered methods to identify and isolate TCRs from neuroprotective T cells from a subject having or suspected of having a central nervous system injury, stroke or neurodegenerative disease. Accordingly, in various embodiments, the isolated TCRs are cloned and incorporated into vectors used the generate modified immune effector cells expressing said TCRs. The modified immune effector cells can then be expanded and administered to the subject.

In one aspect the present disclosure provides methods of identifying a TCR polypeptide chain that can constitute a TCR specific for a CNS peptide antigen useful to direct and/or accumulate immune cells at the site of injury and/or neuronal damage in a subject. To redirect immune effector cells (e.g. T cells) against it sites of neuron damage, T cells can be engineered ex vivo to express CNS-antigen specific T cell receptors (TCRs), generating products referred herein as TCR-engineered immune effector cells (e.g., TCR-T cells).

The method generally comprise the identification and expansion of neuroprotective T cells after CNS injury, stroke or during neurodegenerative. As disclosed herein using an analogs system in mice, T cells are identified as beneficial after CNS injury. Mice without functional T cells result in worsened neuronal survival after injury. Moreover, humans receiving immune suppressive drugs have been shown to recover more poorly after injury and stroke, suggesting immune cells are performing beneficial roles and need to be augmented. The protective cells are most likely autoimmune in nature, hence their selection and expansion are very critical to avoid development of detrimental autoimmune reaction in treated patients.

Injury in the CNS, as in the periphery, results in a cascade of cellular and molecular responses that amplify tissue damage beyond that expected from the severity of the initial injury itself. This process, called secondary degeneration, can lead to severe neurodegeneration even when the initial insult may have only involved partial injury to the nerve or spinal cord. Strikingly, however, the secondary degeneration is more extensive in animals lacking an adaptive immune system than in their wild-type counterparts, suggesting a previously unknown neuroprotective role for immune cells. Restoration of the immune system, and particularly of the T-cell compartment, in immune-deficient mice restores their normal response to CNS injury, further suggesting that an endogenous immune response to CNS injury is neuroprotective. Importantly, it was discovered that not all T cells can mediate this neuroprotective effect, but that the T cells need to be specific to brain-restricted antigens, which governs their migration to, and accumulation in, the injured CNS.

Conventional T cells recognize MHC-presented antigens through their T cell receptor (TCR), a disulfide-linked heterodimer comprised of an α and β chain. To form a functional receptor, TCR α/β heterodimers further complex with CD3ϵ/γ/δ/ζ subunits. TCRs recognize enzymatically cleaved peptides that are presented at the cell surface by MHC molecules (pMHC). In humans, antigen-presenting MHC alleles are broadly classified as HLA class I (A, B, or C) or HLA class II (DR, DP, or DQ), which predominantly present cytosolic or extracellular derived peptides, respectively. The coreceptors CD8 and CD4 enhance TCR antigen sensitivity through interaction with MHC class I or II molecules, respectively. TCR binding to cognate pMHC leads to the phosphorylation of immunoreceptor tyrosine-based activation motifs (ITAMs) in intracellular regions of the CD3 subunits, which results in T cell activation and initiation of effector functions including proliferation, cytokine secretion, and cytolysis via secretion of perforin and granzyme. In TCR-T cell therapy, T cells are edited to express TCR α and β chains that confer a desired specificity. Here, introduced TCR α and β chains dimerize and complex with endogenous CD3 components to form a functional TCR that redirects T cell specificity towards an antigen of interest.

V(D)J recombination of TCRs during thymic development results in a tremendous diversity of TCR sequences within the human T cell repertoire. It is estimated that in an average adult human, there are approximately 4×10¹¹ total circulating T cells and an estimated 10¹⁰ unique T cell clonotypes. Thus, for the vast majority of T cell clones with specificity towards non-viral antigens, the clonal frequency in peripheral blood is far below what is needed to perform the various manipulations required to isolate antigen-specific TCRs given current technologies. Therefore, the present disclosure provides methods for TCR isolation efforts comprising a method step that allows for enrichment of T cells with the desired antigen specificity.

In some embodiments, the methods include obtaining a biological sample comprising neuroprotective T cells from a subject with a CNS injury, stroke, or neurodegeneration. As used herein, a “biological sample” refers to a sample of tissue, cells, or fluid isolated from a subject, including but not limited to, for example, blood, buffy coat, plasma, serum, immune cells (e.g., T cells), CSF, sputa, fecal matter, urine, bone marrow, bile, spinal fluid, lymph fluid, samples of the skin, external secretions of the skin, respiratory, intestinal, and genitourinary tracts, tears, saliva, milk, organs, and biopsies. In a preferred embodiment, the biological sample is a CNS tissue biopsy. In certain embodiments, the CNS tissue biopsy is from a site of injury or disease and comprises infiltrating lymphocytes. At sites of neurodegeneration or injury there is often a large presence of infiltrating lymphocytes. Compared to peripheral blood T cells, T cells within the injured or diseased tissue are often enriched in clones with CNS-antigen specificity.

In some embodiments, the subject is a human. A human subject may be waiting for medical care or treatment, may be under medical care or treatment, or may have received medical care or treatment. In various embodiments, a human subject with a traumatic brain and/or spinal cord injury, may be a subject with signs and/or symptoms of a neurodegenerative disease, or a subject diagnosed with a neurodegenerative disease. In other embodiments, the subject is a laboratory animal.

The biological sample may have been obtained by standard surgical techniques including a biopsy puncture. Multiple biological samples contemporaneously collected from the subject may be pooled. Once collected, the biological samples may have been processed according to methods known in the art (e.g., centrifugation to remove whole cells and cellular debris; use of additives designed to stabilize and preserve the specimen prior to analytical testing; etc.). biological samples may be used immediately or may be frozen and stored indefinitely. Prior to use in the methods disclosed herein, the biological sample may also have been modified, if needed or desired, to include protease inhibitors, detergent(s) and chaotropic agent(s), and/or to deplete other agents (e.g. proteins peptides, metabolites). By way of non-limiting example, a commercially available kit including the EasySep™ Human CD4⁺ T Cell Isolation Kit (Stemcell Technologies) may be used to isolate CD4⁺ T cells from a human blood sample.

Thus, the present disclosure provides method of isolating CNS-antigen-specific T cells from a biological sample obtained from a subject. In some embodiments, the CNS-antigen-specific T cells are expanded ex vivo. Methods for expanding T cells are known in the art and contemplated herein. For example, suitable methods for expanding T cells from a biological sample include those described in Nat Med (2018) 24:724-30. doi: 10.1038/s41591-018-0040-8; N Engl J Med (2016) 375:2255-62. doi: 10.1056/NEJMoa1609279; JNCI J Natl Cancer Inst (1994) 86:1159-66. doi: 10.1093/JNCI/86.15.1159; Nat Rev Cancer (2008) 8:299-308. doi: 10.1038/NRC2355; and BMC Med (2021) 19:1-7. doi: 10.1186/S12916-021-02006-4 and are incorporated by reference in their entirety.

After obtaining polyclonal T cell products that are enriched for T cells with specificities of CNS-antigens, it is necessary to isolate the antigen-specific T cells from the bulk T cell population. In some embodiments, single-cell RNA sequencing is performed on the obtained T cells allowing for single-cell assessment of cellular gene expression, including TCRs, as well as the sequence of gene transcripts. Thus allowing for the identification of CNS-antigen-specific T cells through, in part, their increased expression of effector cytokines such as IFN-γ, TNF-α, and/or IL-2, and from this same data set the sequences of transcripts for the TCR α and β chains from the activated cells.

In another embodiment, obtained T cell populations are analyzed using single cell RT-PCR to amplify TCR α and β chains. In certain embodiments, single T cells are FAC sorted into wells containing RT-PCR reaction buffer, and from a single cell RT-PCR is performed and the TCR α and β chains are PCR amplified. This method reduces the time and labor required for expansion of individual T cell clones; however, a downside to this approach is that confirmatory assays to assess antigen specificity cannot be performed on the T cell clones prior to sequencing.

In another embodiment, the method for obtaining T cell clones is the outgrowth of T cell clones in individual wells. For example, in the limiting dilution method, T cells are diluted to obtain a cell concentration allowing for approximately one cell to be deposited into each well of a plate (e.g., 96-well dish). An alternative method is to FACs sort the T cell population to deliver a single cell into each well. In still another embodiment, each individual T cell may be given a unique barcode using a nucleic acid tag. The goal is to obtain expanded clonal populations of the T cells of interest, which can then be additionally screened for antigen-specificity and sequenced, for example, via Sanger sequencing.

In some embodiments, pathogen reactive T cells are depleted from the T cell populations. Pathogen reactive T cells captured in the biological sample are likely present because they are protecting the tissue from infections and not targeting the injured neurons. Human T cells reactive to pathogens can be predicted based on the TCR sequence using published algorithms for example as described in Nature. 2017 Jul. 6; 547(7661): 94-98 and incorporated herein by reference.

In some embodiments, the obtained biologic sample is assessed for MHC peptidome. In an exemplary embodiment, the MHC peptidome is measured by high-resolution mass spectrometry. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer from ThermoFisher Scientific). Additional processing of the biological sample may occur prior to MS analysis. For example, peptides may be depleted using a size exclusion column and/or proteolytically digested. Suitable proteases include, but are not limited to, trypsin, Lys-N, Lys-C, and Arg-N. Affinity purification may be used to produce an isolated peptide samples, digestion may occur after eluting from the immobilized ligand or while bound. Following one or more clean-up steps, digested peptides may be separated by a liquid chromatography system inter-faced with a high-resolution mass spectrometer. The chromatography system may be optimized by routine experimentation to produce a desired LC-MS pattern. A wide array of LC-MS techniques may be used to analyze the MHC peptidome. Non-limiting examples include selected-reaction monitoring, parallel-reaction monitoring, selected-ion monitoring, and data-independent acquisition. In an exemplary embodiment, a mass spectrometry protocol outlined in the Examples is used.

HLA class I epitopes are peptide fragments, typically 8-12 amino acids in length, generated through processing of ubiquitinated proteins by the proteasome. The proteasome is a large protein complex responsible for the degradation of endogenous proteins that have been damaged or are not needed by the cell and have been tagged by ubiquitin conjugation. The subunits β1, β2, and β5 of the proteasome's 20S catalytic core are associated with the three major catalytic activities of the proteasome. While proteasomes that incorporate subunits β1, β2, and β5 are referred to as the ‘standard proteasome’, hematopoietic cells and cells stimulated with certain inflammatory cytokines (e.g., INF-γ, IFN-α, IFN-β, and TNF-α) alternatively express β1i, β2i, and β5i subunits that displace β1, β2, and β5 subunits in the proteasome, forming an isoform termed the ‘immunoproteasome’. The immunoproteasome displays several biochemical differences that influence peptide cleavage activity. This results in the immunoproteasome producing peptide products with enhanced immunogenicity compared to the standard proteasome, as these immunoproteasome-generated peptides are more likely to contain C-terminal hydrophobic residues, which are associated with more efficient HLA-class I binding. In addition, there are ‘intermediate proteasomes’ that contain a mixture of standard and immunoproteasome subunits, specifically substituting only β5i or β1i plus β5i and result in a peptide repertoire similar to that produced by the immunoproteasome, but includes additional unique peptide products.

Once identified, TCR α and β chains of T cells of interest are then cloned, in a non-limiting example, from cDNA through PCR amplification. However, a unique challenge is that their 5′ regions are highly variable. To overcome this, one of two PCR variations can be employed, 5′ RACE or multiplex PCR. Thus, the present disclosure provides nucleic acid sequences encoding the identified TCR α and β chains of interest and vectors comprising the same. A nucleic acid encoding a TCR polypeptide chain, optionally alpha or beta, or delta or gamma that has, in an embodiment, been previously isolated (e.g. identified and cloned as described above) and/or for which the sequence of the CDR regions (e.g. CDR1, CDR2 and CDR3) have been determined. For example, for TCR chains wherein the sequence of the CDR regions have been determined, a nucleic acid can be cloned and/or constructed, optionally by combining recombinantly produced CDR region nucleic acids with a known constant region, and/or replacing CDR regions in a known TCR chain (e.g. known sequence). Methods for TCR cloning, delivery, expression, and the manufacturing of clinical-grade TCR expressing immune cell populations are known in the art.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

A further object of the present invention relates to a nucleic acid sequence that encodes for the amino acid sequence of the α chain and/or the β chain, or functional part thereof (e.g., CDRs) of identified by the methods of the present disclosure.

As used herein, the term “nucleic acid sequence” has its general meaning in the art and refers to a DNA or RNA sequence. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the nucleic acid sequence may comprise both the TCR α and TCR β chains linked with a self-cleaving peptide sequence (e.g., P2A). TCR construct of the disclosure may further comprise a detectable label used to sort cells expressing the heterologous TCRs (e.g., peptide tags or fluorescent proteins). TCR α/β chains form heterodimers largely through interactions within TCR constant regions. Endogenous TCR α/β chains form a disulfide bond between TCR α constant region (Cα) residue 94 and TCR β constant region (Cβ) residue 130. The proper pairing of introduced TCRs can be improved by introducing a second stabilizing disulfide bond through cysteine substitutions at Cα residue 48 and Cβ residue 57, which increases interchain binding affinity of introduced TCR α/β chains while decreasing binding affinity with endogenous TCR α/β chains.

The endogenous TCR α chain has a relatively low stability, which can be increased by substituting leucine and valine residues within the Ca transmembrane region. TCR α chains containing these stabilizing mutations, termed α-LVL, demonstrate increased TCR surface expression and biological activity. While this strategy promotes pairing of an introduced TCR by stabilizing the TCR α chain, the TCR β chain remains unmodified and thus susceptible to mispairing. However, this can be addressed by incorporating the α-LVL substitutions into murinized TCRs, the combination of which can synergistically enhance TCR expression and biological activity.

Through TCR crystal structure analysis, several amino acids mediating TCR α/β dimerization have been identified. By swapping two such interacting residues, a Ca glycine and Cβ arginine, mutant TCR α/β chains can be generated with a similar propensity for dimerizing with each other, but with a significantly reduced propensity to bind with unmodified endogenous TCR α/β chains. Other examples of TCR domain swapping/conjugation strategies include swapping with γδ TCR constant regions, replacing regions with CD3ζ (153, 154) or CD28/CD3ϵ (155), or conjugation to leucine zipper dimerization motifs.

To combine the antigen recognition properties of a TCR α/β heterodimer into a single chain, several groups have developed so-called three-domain single-chain TCRs (scTCR), which are composed of Vα/Vβ regions fused by a short peptide linker and conjugated to a Cβ domain. To mediate signal transduction, three-domain scTCRs are typically further conjugated to CD3ζ. scTCR constructs utilizing CD3ζ transmembrane and signaling domains function independently of the CD3 complex, which theoretically allows for higher surface expression to be achieved with scTCRs than with native TCRs, as scTCRs are not limited by the abundance of CD3 components. The CD3-independence of scTCRs may also be beneficial in applications where it is desirable to maintain levels of endogenous TCR expression. Rather than modifying the introduced TCR, other strategies address mispairing through knock-down or knock-out of the endogenous TCR.

DNA molecules encoding TCRs can be chemically synthesized. Synthetic DNA molecules can be ligated to other appropriate nucleotide sequences, including, e.g., constant region coding sequences, and expression control sequences, to produce conventional gene expression constructs encoding the desired TCR. Production of defined gene constructs is within routine skill in the art.

Nucleic acids encoding desired antibodies can be incorporated (ligated) into expression vectors, which can be introduced into host cells through conventional transfection or transformation techniques.

Specific expression and purification conditions will vary depending upon the expression system employed. If the engineered gene is to be expressed in eukaryotic host cells, e.g., T cells, it is first inserted into an expression vector containing a suitable eukaryotic promoter, a secretion signal, a poly A sequence, and a stop codon, and, optionally, may contain enhancers, and various introns. This expression vector optionally contains sequences encoding all or part of a constant region, enabling an entire, or a part of, a TCR to be expressed. The gene construct can be introduced into eukaryotic host cells using conventional techniques. In some embodiments, a host cell is transfected with a single vector expressing a polypeptide expressing an entire, or part of, a TCR. In other embodiments, a host cell is transfected with a single vector encoding (a) a polypeptide comprising a TCR chain, or (b) an entire TCR. In still other embodiments, a host cell is co-transfected with more than one expression vector (e.g., one expression vector encoding a polypeptide comprising an entire, or part of, a TCR chain, and another expression vector encoding a polypeptide comprising an entire, or part of, another TCR chain).

Therefore, in various embodiments, a nucleic acid is provided, the nucleic acid comprising a nucleotide sequence encoding the TCR described herein. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Suitable nucleic acids that can encode portions of the inventive TCRs can be determined using standard techniques. In various embodiments, the nucleic acid comprises a nucleotide sequence encoding a TCR identified by the methods described herein. In some embodiments, the nucleic acids encode one or more complementary determining regions (CDR) having the amino acid sequences identified by the methods described herein.

In various embodiments, an expression vector is provided comprising one or more of the nucleic acids described herein. Vectors can be derived from plasmids such as: F, F1, RP1, Col, pBR322, TOL, Ti, etc; cosmids; phages such as lambda, lambdoid, M13, Mu, P1, P22, Q3, T-even, T-odd, T2, T4, T7 etc; or plant viruses. Vectors can be used for cloning and/or expression of the binding molecules of the invention and might even be used for gene therapy purposes. Vectors comprising one or more nucleic acid molecules according to the invention operably linked to one or more expression-regulating nucleic acid molecules are also covered by the present invention. The choice of the vector is dependent on the recombinant procedures followed and the host used. Introduction of vectors in host cells can be affected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the human binding molecules as described above operably linked to one or more nucleic acid molecules encoding proteins or peptides that can be used to isolate the human binding molecules are also covered by the invention. These proteins or peptides include, but are not limited to, glutathione-S-transferase, maltose binding protein, metal-binding polyhistidine, green fluorescent protein, luciferase and beta-galactosidase.

The expression vector may be transfected into a host cell to induce the translation and expression of the nucleic acid into the heavy chain variable region and/or the light chain variable region. Therefore, a host cell is provided comprising any expression vector described herein. Host cells include, but are not limited to, cells of mammalian, plant, insect, fungal or bacterial origin. Bacterial cells include, but are not limited to, cells from Gram-positive bacteria or Gram-negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by methods such as Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells, such as Chinese Hamster Ovary (CHO) cells, COS cells, BHK cells, NSO cells or Bowes melanoma cells are preferred in the present invention. Since the present invention deals with molecules that may have to be administered to humans, a completely human expression system would be particularly preferred. Therefore, even more preferably, the host cells are human cells. Examples of human cells are, inter alia, HeLa, 911, AT1080, A549, HEK293, 293F and HEK293T cells.In some embodiments, the nucleic acid sequence of the present disclosure is included in a suitable vector, such as a plasmid, cosmid, episome, artificial chromosome, phage or a viral vector. Hence, a further object of the invention relates to a vector comprising a nucleic acid sequence of the present invention. Typically, the vector is a viral vector which is an adeno-associated virus (AAV), a retrovirus, bovine papilloma virus, an adenovirus vector, a lentiviral vector, a vaccinia virus, a polyoma virus, or an infective virus. In some embodiments, the vector is an AAV vector. As used herein, the term “AAV vector” means a vector derived from an adeno-associated virus serotype, including without limitation, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Retroviruses may be chosen as gene delivery vectors due to their ability to integrate their genes into the host genome, transferring a large amount of foreign genetic material, infecting a broad spectrum of species and cell types and for being packaged in special cell lines. In order to construct a retroviral vector, a nucleic acid encoding a gene of interest is inserted into the viral genome in the place of certain viral sequences to produce a virus that is replication-defective. In order to produce virions, a packaging cell line is constructed containing the gag, pol, and/or env genes but without the LTR and/or packaging components. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV 1, HIV 2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are known in the art, see, e.g. U.S. Pat. Nos. 6,013,516 and 5,994,136, both of which are incorporated herein by reference. In general, the vectors are plasmid-based or virus-based, and are configured to carry the essential sequences for incorporating foreign nucleic acid, for selection and for transfer of the nucleic acid into a host cell. The gag, pol and env genes of the vectors of interest also are known in the art. Thus, the relevant genes are cloned into the selected vector and then used to transform the target cell of interest. Recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. This describes a first vector that can provide a nucleic acid encoding a viral gag and a pol gene and another vector that can provide a nucleic acid encoding a viral env to produce a packaging cell. Introducing a vector providing a heterologous gene into that packaging cell yields a producer cell which releases infectious viral particles carrying the foreign gene of interest. The env preferably is an amphotropic envelope protein which allows transduction of cells of human and other species. Typically, the nucleic acid molecule or the vector of the present invention include “control sequences’”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is repressed? by an analyte, cofactor, regulatory protein, etc.), and “constitutive promoters”.

A further object of the present disclosure relates to a modified immune effector cell which comprises a nucleic acid sequence identified using the method of the present disclosure. In some embodiments, the cell expresses the T-cell receptor of the present disclosure.

In some embodiments, the cell is a T-cell (e.g., CD4⁺ or CD8⁺). The cell may be derived from a T-cell isolated from a subject. The T-cell may be part of a mixed cell population isolated from the subject, such as a population of peripheral blood lymphocytes (PBL) or whole unfractionated blood. T cells within the PBL population may be activated by methods known in the art, such as using anti-CD3 and CD28 antibodies or antigen-specific stimulation with peptide-pulsed antigen presenting cells. The T-cell may be a CD4⁺ helper T cell or a CD8⁺ cytotoxic T cell. The cell may be in a mixed population of CD4⁺ helper T cells/CD8⁺ cytotoxic T cells. Polyclonal activation, for example using anti-CD3 antibodies optionally in combination with anti-CD28 antibodies or mitogens such as phytohemagglutinin together with suitable cytokine cocktails will trigger the proliferation of CD4⁺ and CD8⁺ T cells, but may also trigger the proliferation of CD4⁺ CD25⁺ regulatory T-cells.

A further object of the present disclosure relates to a method of producing the cell of the present disclosure, which comprises the step of transfecting or transducing a cell in vitro or ex vivo with the vector of the present invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. In some embodiments, the nucleic acid encoding a TCR, or functional fragment thereof, is mRNA and the host cell transformed by electroporation or by liposome mediated delvery.

In some embodiments, the cell is isolated from a subject to whom the genetically modified cell is to be adoptively transferred. In some embodiments, a population of cells of the present invention are obtained by isolating a population of T-cells from a subject, optionally expanding said population of T cells in a population of T cells, and by subsequently proceeding with TCR gene transfer ex vivo and subsequent immunotherapy of the subject by adoptive transfer of the TCR-transduced cells. Alternatively, the population of cells is isolated from a different subject, such that it is allogeneic. In some embodiments, the population of cells is isolated from a donor subject. Alternatively the population of cells is, or is derived from, a population of stem cells, such as a haemopoietic stem cells (HSC). Gene transfer into HSCs does not lead to TCR expression at the cell surface, as stem cells do not express the CD3 molecules. However, when stem cells differentiate into lymphoid precursors that migrate to the thymus, the initiation of CD3 expression leads to the surface expression of the introduced TCR in thymocytes. An advantage of this approach is that the mature T cells, once produced, express only the introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T-cells with the desired antigen specificity. The cell may therefore be a gene-modified stem cell, which, upon differentiation, produces a T-cell expressing a TCR of the present invention. The present disclosure also relates to a method of producing a T-cell expressing a TCR of the present disclosure by inducing the differentiation of a stem cell which comprises a nucleotide sequence of the present invention. Any carrier cell suitable for accepting the introduced TCR and expressing it in functional form can be used for research or therapeutic purposes. Further examples of such cells include, but are not limited to, Jurkat cells, T-cell hybridomas, lines or clones. All these cells may be expressing or not their endogenous TCRs.

Once a population of CNS-antigen specific TCR-expressing immune effector cells are generated the can be further selected to focus on CNS specific and CNS enriched peptides. CNS specific and enriched peptides with low or no predicted binding to the subject's MHC will be synthesized. A mixture of these peptides will be incubated to assess CNS-antigen specific TCR-expressing immune effector cells specificity, in a non-limiting example, using multi-well IL-2 ELISpot or ELISA assay. T cells that do not react to any CNS antigens will be eliminated. The responding clones will be graded from low to high affinity based on IL-2 release. Approaches in this regard typically involve stimulating CNS-antigen specific TCR-expressing immune effector cells with the cognate antigen of interest, and then isolating antigen-responsive T cells based on increased expression of known activation-associated molecules. This includes antibody staining of transmembrane proteins that are transiently upregulated following T cell stimulation (e.g., 4-1 BB and OX40 in CD8⁺ and CD4⁺ T cells, respectively), allowing for isolation of these cells by FAC sorting or magnetic bead separation. Another approach is IFN-γ-capture, whereby antigen stimulated T cells are identified and captured based on production of IFN-γ, which is rapidly secreted by antigen-stimulated CD8⁺ and Th1 CD4⁺ T cells.

In some embodiments, CNS-antigen specific TCR-expressing immune effector cell clones with low specificity to CNS antigens are expanded in vitro for administration into the subject.

The population of cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular antigen are desired, then the population will contain greater than 70%, generally greater than 80%, 85% and 90-95% of such cells. The desired purity can be achieved by introducing a sorting step following introduction of the desired TCR sequence using methods such as HLA multimers and others known in the art. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

Typically, the population of cells of the present disclosure is administered to the subject in the form of pharmaceutical composition. The pharmaceutical composition may be produced by those of skill, employing accepted principles of treatment. Such principles are known in the art, and are set forth, for example, in Braunwald et al., eds., Harrison's Principles of Internal Medicine, 19th Ed., McGraw-Hill publisher, New York, N.Y. (2015), which is incorporated by reference herein. The pharmaceutical composition may be administered by any means that achieve their intended purpose. For example, administration may be by parenteral, subcutaneous, intravenous, intradermal, intramuscular, intraperitoneal, transdermal, or buccal routes. The pharmaceutical compositions may be administered parenterally by bolus injection or by gradual perfusion over time. The pharmaceutical compositions typically comprises suitable pharmaceutically acceptable carriers comprising excipients and auxiliaries which may facilitate processing of the active compounds into preparations which can be used pharmaceutically. The pharmaceutical compositions may contain from about 0.001 to about 99 percent, or from about 0.01 to about 95 percent of active compound(s), together with the excipient.

Pharmaceutically acceptable excipients are identified, for example, in The Handbook of Pharmaceutical Excipients, (American Pharmaceutical Association, Washington, D.C., and The Pharmaceutical Society of Great Britain, London, England, 1968). Additional excipients can be included in the pharmaceutical compositions of the invention for a variety of purposes. These excipients can impart properties which enhance retention of the compound at the site of administration, protect the stability of the composition, control the pH, facilitate processing of the compound into pharmaceutical compositions, and so on. Other excipients include, for example, fillers or diluents, surface active, wetting or emulsifying agents, preservatives, agents for adjusting pH or buffering agents, thickeners, colorants, dyes, flow aids, nonvolatile silicones, adhesives, bulking agents, flavorings, sweeteners, adsorbents, binders, disintegrating agents, lubricants, coating agents, and antioxidants.

Accordingly, the present disclosure provides a process of treating, preventing, or reversing a CNS injury, stroke, or neurodegeneration in a subject in need by administration of a therapeutically effective amount of CNS-antigen specific TCR-expressing immune cells, so as to prevent, reduce, or reverse the CNS injury and/or neuronal cell death.

As used herein, a neuronal cell, may also be referred to as a neuron or a nerve cell, is an electrically excitable cell that processes and transmits information through electrical and chemical signals. Neurons are the core components of the brain and spinal cord of the central nervous system (CNS), and of the ganglia of the peripheral nervous system (PNS). Specialized types of neurons include: sensory neurons which respond to touch, sound, light and all other stimuli affecting the cells of the sensory organs that then send signals to the spinal cord and brain, motor neurons that receive signals from the brain and spinal cord to cause muscle contractions and affect glandular outputs, and interneurons which connect neurons to other neurons within the same region of the brain, or spinal cord in neural networks.

Neuroprotection may be determined by measuring cell death of neuronal cells. Methods of measuring cell death are known in the art. For example, cell death may be measured by Giemsa staining, trypan blue exclusion, acridine orange/ethidium bromide (AO/EB) double staining for fluorescence microscopy and flow cytometry, propidium iodide (PI) staining, annexin V assay, TUNEL assay, DNA ladder, LDH activity, and MTT assay. Cell death may be due to induction of apoptosis. Cell death due to induction of apoptosis may be measured by observation of morphological characteristics including cell shrinkage, cytoplasmic condensation, chromatin segregation and condensation, membrane blebbing, and the formation of membrane-bound apoptotic bodies. Cell death due to induction of apoptosis may be measured by observation of biochemical hallmarks including internucleosomal DNA cleavage into oligonucleosome-length fragments. Traditional cell-based methods of measuring cell death due to induction of apoptosis include light and electron microscopy, vital dyes, and nuclear stains. Biochemical methods include DNA laddering, lactate dehydrogenase enzyme release, and MTT/XTT enzyme activity. Additionally, terminal deoxynucleotidyl transferase-mediated dUTP-biotin nick end labeling of DNA fragments (TUNEL) and in situ end labeling (ISEL) techniques are used, which when used in conjunction with standard flow cytometric staining methods yield informative data relating cell death to various cellular parameters, including cell cycle and cell phenotype. See Loo and Rillema, Methods Cell Biol. 1998; 57:251-64, which is incorporated herein by reference, for a review of these methods.

Neuroprotection may be determined by reducing the signs or symptoms associated with stroke. For example, signs or symptoms of associated with a stroke include trouble with speaking and understanding; paralysis or numbness of the face, arm, or leg; trouble with seeing in one or both eyes; headache(s); trouble with walking; etc.

Neuroprotection may be determined by reducing the signs or symptoms associated with a neurodegenerative disease. For example, signs or symptoms associated with a neurodegenerative disease include memory loss; loss of control in walking, balance, mobility, vision, speech, and swalling; loss of behavior control, emotion, and language; etc.

The results of these methods may be used to determine the percentage of viable cells. In an embodiment, cell death may be measured as a reduction in viable cells. Since a composition of the disclosure decreases neuronal cell death, an increase in viable cells relative to untreated neuronal cells undergoing cell death is indicative of decreasing neuronal cell death. As such, an increase in viable cells following administration of CNS-antigen specific TCR-expressing immune cells may be greater than 1% relative to untreated neuronal cells undergoing cell death. For example, an increase in viable cells may be greater than 1%, greater than 2%, greater than 3%, greater than 4%, or greater than 5% relative to untreated neuronal cells undergoing cell death. Alternatively, an increase in viable cells may be greater than 5%, greater than 6%, greater than 7%, greater than 8%, greater than 9%, or greater than 10% relative to untreated neuronal cells undergoing cell death. Additionally, an increase in viable cells may be greater than 10%, greater than 11%, greater than 12%, greater than 13%, greater than 14%, or greater than 15% relative to untreated neuronal cells undergoing cell death. Further, an increase in viable cells may be greater than 15%, greater than 20%, greater than 25%, greater than 30%, greater than 35%, greater than 40%, greater than 45%, or greater than 50% relative to untreated neuronal cells undergoing cell death. Still further, an increase in viable cells may be greater than 50% greater than 55%, greater than 60%, greater than 65%, greater than 70%, greater than 75%, or greater than 80%, greater than 85%, or greater than 90%, or greater than 95% relative to untreated neuronal cells undergoing cell death.

In another embodiment, an increase in viable cells relative to untreated neuronal cells undergoing cell death is measured using p-value. For instance, when using p-value, an increase in viable cells relative to untreated neuronal cells undergoing cell death following administration of CNS-antigen specific TCR-expressing immune cells occurs when the p-value is less than 0.1, preferably less than 0.05, more preferably less than 0.01, even more preferably less than 0.005, the most preferably less than 0.001.

In certain embodiments, the neuronal cell death is due to ischemia. In other embodiments, the neuronal cell death is due to stroke.

In still another aspect, the disclosure provides a method of treating or preventing stroke. The method comprises administering a composition comprising CNS-antigen specific TCR-expressing immune cells. A stroke occurs when the blood supply to part of the brain is interrupted or severely reduced, depriving brain tissue of oxygen and nutrients. A suitable subject may or may not be at risk for a stroke. Non-limiting examples of risk factors for stroke include overweight or obese, physical inactivity, heavy or binge drinking, use of illicit drugs such as cocaine and methamphetamines, high blood pressure, cigarette smoking or exposure to second hand smoke, high cholesterol, diabetes, obstructive sleep apnea, cardiovascular disease including heart failure, heart defects, heart infection or abnormal heart rhythm, personal or family history of stroke, heart attack or transient ischemic attack, 55 or older, race (African Americans have a higher risk), gender (men have a higher risk). A suitable subject may or may not have a sign or symptom associated with stroke. Non-limiting examples of signs or symptoms associated with stroke include trouble speaking and understanding, paralysis or numbness of the face, arm or leg, trouble seeing in one or both eyes, headache, and/or trouble with walking. Specifically, the stroke may be ischemic stroke. Ischemic stroke may be thrombotic stroke or embolic stroke. Additionally, the stroke may be a transient ischemic attack (TIA), also referred to as a ministroke.

In still yet another aspect, the disclosure provides a method of treating or preventing a disease associated with neuronal cell degeneration. In an embodiment, the disclosure provides a method of treating or preventing a neurodegenerative disease. As used herein, a “neurodegenerative disease” is a term for a range of conditions that primarily affect the neurons of the nervous system resulting in degeneration and/or death of nerve cells. Non-limiting examples of neurodegenerative diseases include amyotrophic lateral sclerosis, Parkinson's disease, Alzheimer's disease, Huntington's disease, motor neuron diseases, spinocerebellar ataxia, spinal muscular atrophy, and prion disease. Non-limiting examples of diseases or disorders that may be associated with neuronal cell death or degeneration include schizophrenia, depression, bipolar disorder (Type I or Type II), schizoaffective disorder, mood disorders, anxiety disorders, personality disorders, psychosis, compulsive disorders, post-traumatic stress disorder (PTSD), Autism spectrum disorder (ASD), dysthymia (mild depression), social anxiety disorder, obsessive compulsive disorder (OCD), pain (e.g., a painful syndrome or disorder), sleep disorders, memory disorders (e.g., memory impairment), dementia, Alzheimer's Disease, a seizure disorder (e.g., epilepsy), traumatic brain or spinal cord injury, stroke, addictive disorders (e.g., addiction to opiates, cocaine, and/or alcohol), autism, Huntington's Disease, insomnia, Parkinson's disease, withdrawal syndromes, and tinnitus.

In some embodiments, the composition further comprises at least one other therapeutic, prophylactic and/or diagnostic agent. Preferably, the therapeutic and/or prophylactic agents are capable of preventing and/or treating a CNS injury, stroke or neurodegeneration and/or a condition/symptom resulting from the same. Therapeutic and/or prophylactic agents include, but are not limited to, neuroprotective agents. Such agents can be binding molecules, small molecules, organic or inorganic compounds, enzymes, polynucleotide sequences, peptides, etc.

The additional therapeutic/prophylactic and/or diagnostic agents may be used in combination with the modified cells of the present invention. “In combination” herein, means simultaneously, as separate formulations (e.g., co-administered), or as one single combined formulation or according to a sequential administration regiment as separate formulations, in any order. Agents capable of preventing and/or treating a CNS injury, stroke or neurodegeneration and/or a condition resulting from the same that are in the experimental phase might also be used as other therapeutic and/or prophylactic agents useful in the present invention.

Dosing regiments can be adjusted to provide the optimum desired response (e.g., a prophylactic or therapeutic response). Therefore, the dose used in the methods herein can vary depended on the intended use (e.g., for prophylactic vs. therapeutic use). Furthermore, a single bolus may be administered, several divided doses may be administered over time, or the dose may be proportionally reduced or increased as indicated by the exigencies of the therapeutic of the therapeutic situation.

Generally, the methods as described herein comprise administration of a therapeutically effective amount of a composition of the disclosure to a subject. The methods described herein are generally performed on a subject in need thereof. A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Personalized Cell Based Therapy for CNS Injury

Spinal cord injury, mainly induced by motor vehicle accidents and accidental falls, makes millions of people worldwide suffer from lifelong disability of muscle movement, sensation and even breathing. The lack of efficient treatment makes spinal cord injury a severe burden for both individuals and healthcare system. The feature of immune cells that can migrate to the injury site after injury inspire scientists using immune cell as carrier to develop cellular therapy. Antigen specificity of T cells make it an excellent candidate and CAR-T therapy have already been shown to be effective in cancer therapy. Previous studies have found CD4⁺ T cell have neuro-protective function but data is lacking regarding the molecular mechanism required for T cell mediated neuroprotection. The present Example used single cell TCR sequencing to get self-generated auto-immune TCRs and generate TCR based CD4⁺ T cell therapy for CNS injury. This present example provides a proof or principle strategy which can be used to identify the molecular target specific for a subject's injury or disease and production of a personalized T cell therapy for that subject.

Our MHC-II complex immunoprecipitation data shows that after spinal cord injury, abundance of self-peptides can be presented on MHC-II molecules and single cell sequencing data of T cell in the injury site shows high clonality of these tissue infiltrated T cells, which means that T cells in the injury site have already met self-antigen produced by injured tissue and have been activated.

Different from CD8⁺ T cells which have cytotoxic capacity, the present example found that auto-immune CD4⁺ T cells, in the early stage of CNS injury, have beneficial effect. By reconstitution of auto-immune TCRs derived from sequencing of primary CD4⁺ T cells isolated and enriched from the injury site, administration of CNS-antigen specific TCR-expressing T cell therapy worked in the treatment and prevention of neuronal cell death for both optic nerve injury and spinal cord injury. To avoid the potential risk that auto-immune T cell may induce auto-immune disease in the later stage of recovery, an mRNA based transient TCR expression is used and shown to be successful. Treatment of mRNA-TCR based therapeutic T cells after spinal cord injury highly improved locomotion skills of spinal cord injury mice. Auto-immune T cells may induce auto-immune response but mRNA-TCR based T cell therapy helped mice recover well after CNS injury with minimized side effect. The beneficial effect of therapeutic T cell was also reflected histologically with the smaller and more contained scar in the injury site.

NFAT-GFP reporter hybridoma system helped us confirm the antigen of the TCR derived from TCR sequencing. Many of the derived TCR clones, like Du, Eo, Fl and Go, were shown to respond to CNS enriched protein MOG and Cp responded to Licam. By checking the injury site of spinal cord injury in mice with/out therapeutic T cell treatment, it was found that therapeutic T cell modulate spinal cord scar by tuning myeloid cell population. Single cell sequencing data of CD45⁺ population in the injury site shows that after therapeutic CNS-antigen specific TCR-expressing T cell treatment, resulted in reduced macrophage migration to the injury site. Moreover, by subtyping the two major population in the injury site, microglia and macrophage, it was found that therapeutic CNS-antigen specific TCR-expressing T cells made microglia less activated and made the macrophage population less inflammatory, more anti-inflammatory and more angiogenic. These chilled macrophage/microglia, in turn, induce less further damage of neuron and helped mice recover better.

In this example, a TCR based auto-immune CD4⁺ T cell therapy was generated and mRNA based TCR expression used to minimize side effects. It is the first time CNS-antigen specific TCR-expressing T cell therapy was used to treat CNS injury and the robust beneficial effects of this CNS-antigen specific TCR-expressing T cell therapy in mice make establish these methods have strong potential in clinic transformation and expand the strategy targeting other CNS disorder.

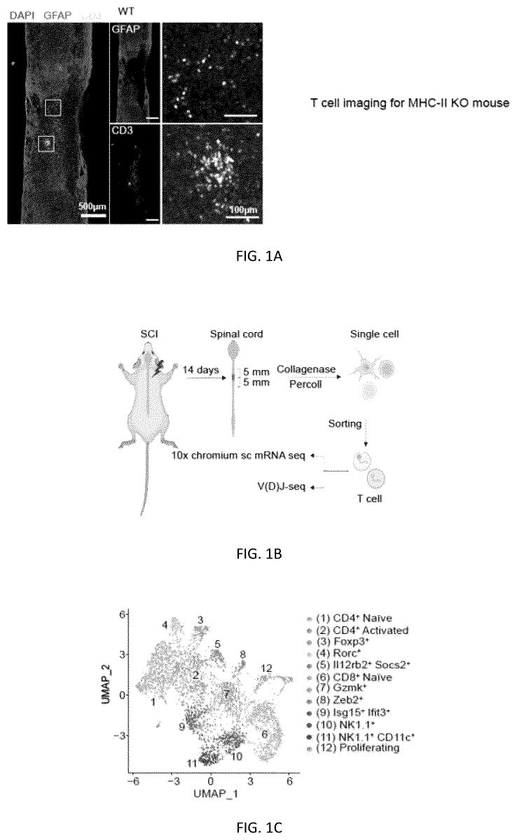

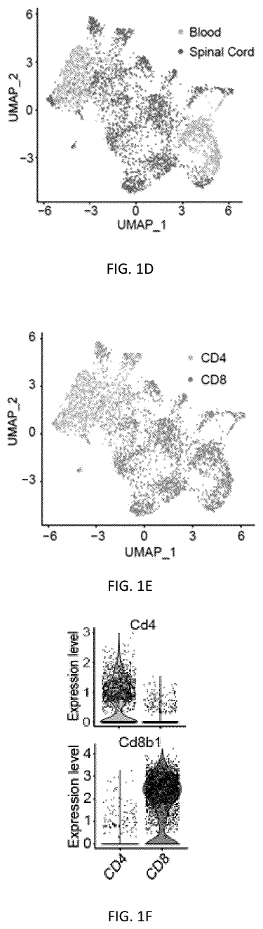

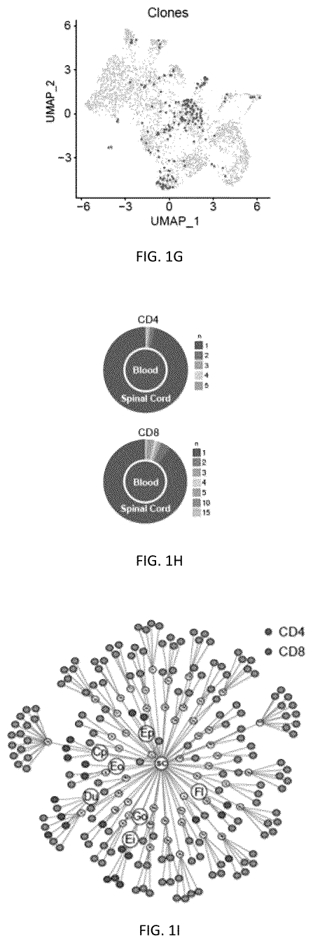

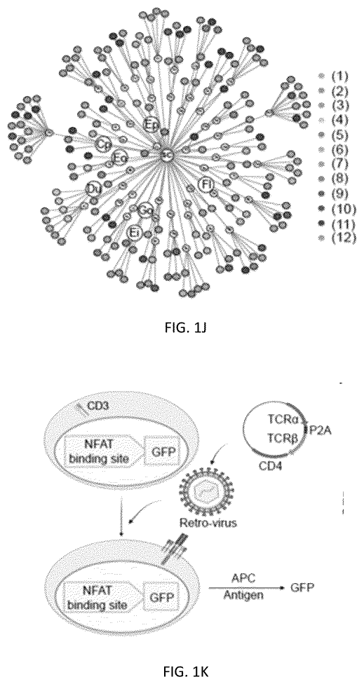

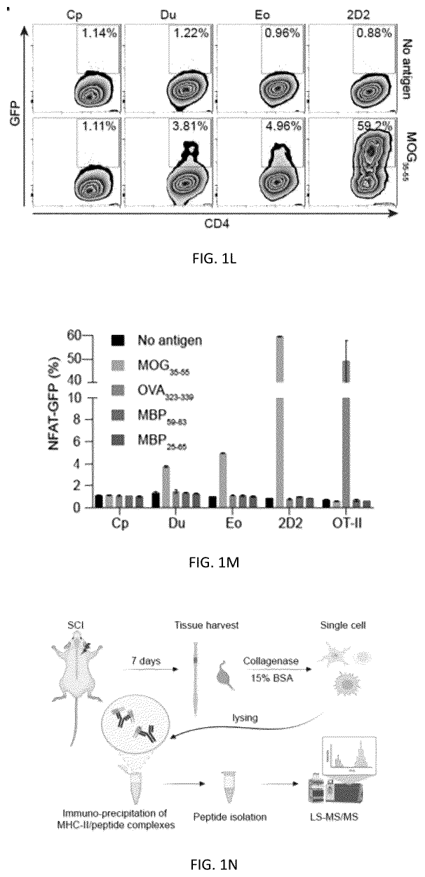

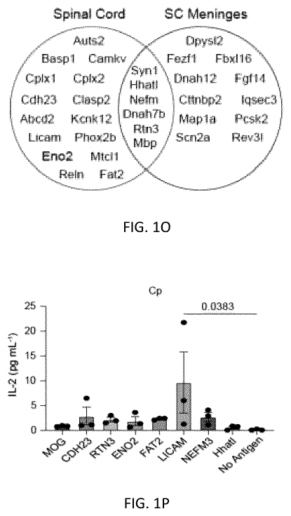

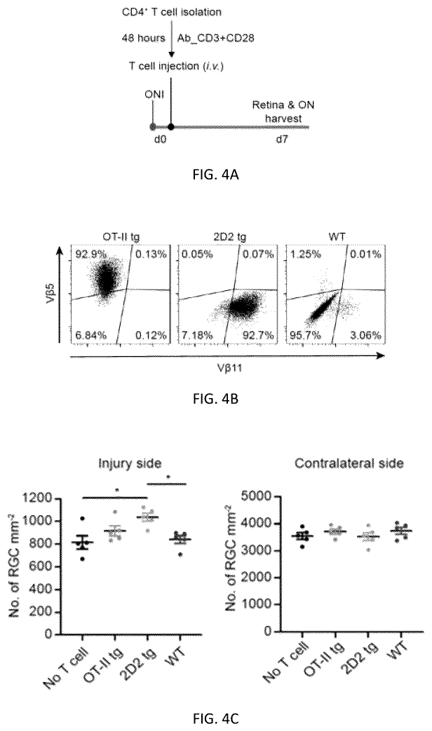

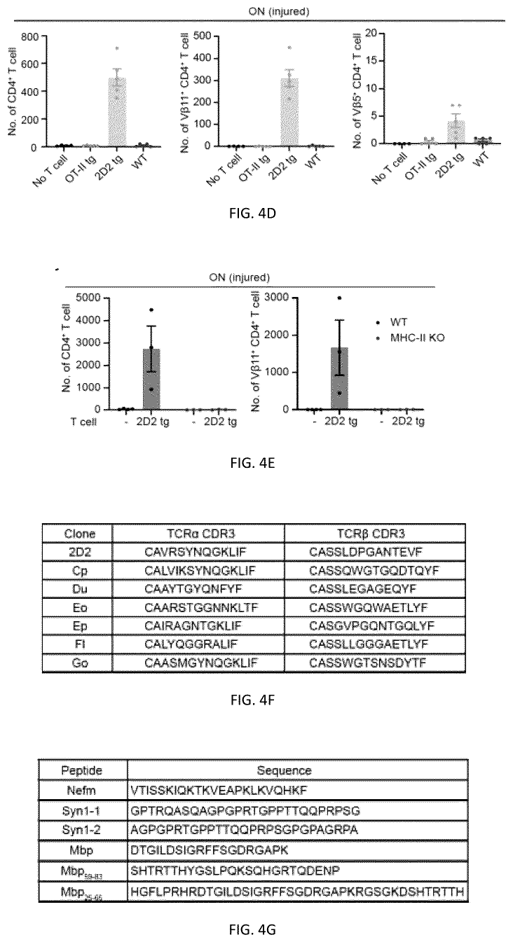

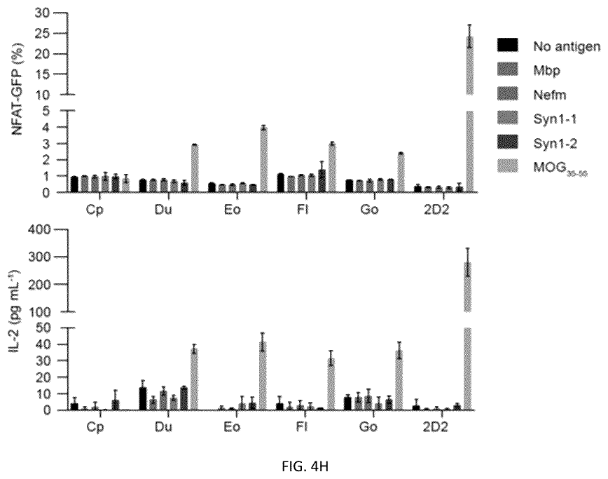

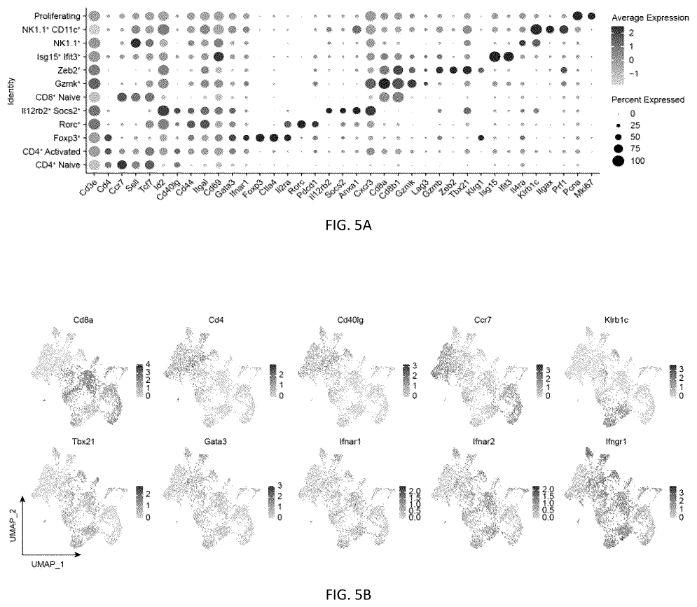

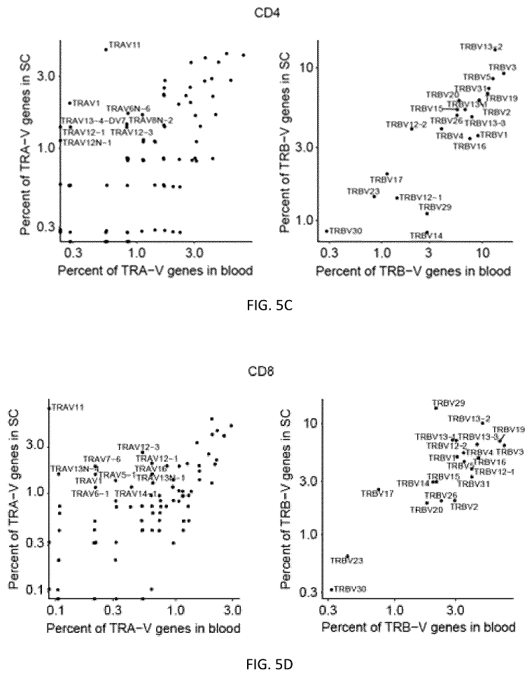

Clonality of auto-immune T cells after CNS injury: By GFAP staining, injury site after spinal cord injury is shown. T cells infiltrated to the parenchyma of spinal cord and most of them enriched in the injury site and this infiltration is depended on MHC-II signaling (FIG. 1A, FIG. 4E). To test if T cell infiltration is driven by antigen specificity, conventional T cells from injured spinal cord were sorted and profiled using single cell 10× chromium sequencing and V(D)J sequencing, with T cells from blood of naïve mice as control (FIG. 1B). Unsupervised clustering using the uniform manifold approximation and projection (UMAP) for dimension reduction revealed 13 different clusters (FIG. 1C, FIG. 5A). T cells from homeostatic blood present naïve T cell features and T cells from injured spinal cord were more activated and differentiated (FIG. 1D). Infiltrating T cells did not cluster with classic T cell subtyping because compared with specific transcription factor, there were additional genes that were different between clusters (FIG. 5B). T cell receptor (TCR) is the key factor to decide antigen specification of T cell and the first step to control T cell activation and proliferation. TCRα chain and TCRβ chain, together with CD3 complex, assemble the functional TCR complex. TCR recombination ensure the diversity of TCRs and CDR3 region in variable chain of both TCRα and TCRβ is believed to be the most diverse and the region to interact with TCR epitope. By analyzing sequences of CDR3 region of result from V(D)J sequencing, if more than two cells sharing the same sequence of TCRα and TCRβ pair, this was defined as a TCR clone. After spinal cord injury, there were much more CD8⁺ T cell clones than CD4⁺ T cell clones (FIG. 1E, FIG. 1F, FIG. 1G, and FIG. 1H), and the clonality of CD8⁺ T cells were much higher than CD4⁺ T cells (FIG. 1I, FIG. 1J), which fit characteristic of T cells that CD8⁺ T cells have higher proliferation capacity after activation and have strong cytotoxic effect to attach target cells. Comparing with T cells from injured spinal cord and homeostatic blood, there were some specific TCRα subtype like TCAV11 is enriched in spinal cord T cells but no specific TCRβ subtype is enriched in spinal cord T cell sample (FIG. 5C). Different from CD8⁺ T cells, function of CD4⁺ T cells after CNS disorder is quite controversial. Overactivation of self-targeting CD4⁺ T cells induce auto-immune disease but there are also publications reported that CD4⁺ T cell has protective function after CNS injury like optic nerve injury and spinal cord injury. Using optic nerve injury model, by injecting CD4⁺ T cells isolated from OT-II, 2D2 TCR transgenic mice or wild type mice, it was found that only 2D2 T cells, which recognize myelin component MOG, protected mice which were shown to recover better from injury (FIG. 4A, FIG. 4B, FIG. 4C). Auto-immune CD4⁺ T cells infiltrated to injured tissue after injury and the infiltration was dependent on MHC-II (FIG. 4D, FIG. 4E).

T cell epitope needs to be presented by antigen presenting cells on MHC-II molecules. MHC-II complex was isolated from injured spinal cord and Mass Spec used to identify a pool of self-peptides that were presented on MHC-II. These peptides correspond to those that induce T cell responses after injury (FIG. 1N, FIG. 1O). NFAT-GFP reporter hybridoma system helped to confirm the antigen of the TCR derived from TCR sequencing. By co-culturing TCR expressing hybridoma with antigen presentation cells and antigen candidates, antigens for specific TCRs were screened by testing IL-2 secretion and GFP signal (FIG. 1K). Most of the identified CD4 clones, like Du, Eo, Fl and Go, were shown to recognize MOG peptide, and clone Cp recognized Licam (FIG. 1I, FIG. 1M, FIG. 1P, FIG. 4G, FIG. 4H).

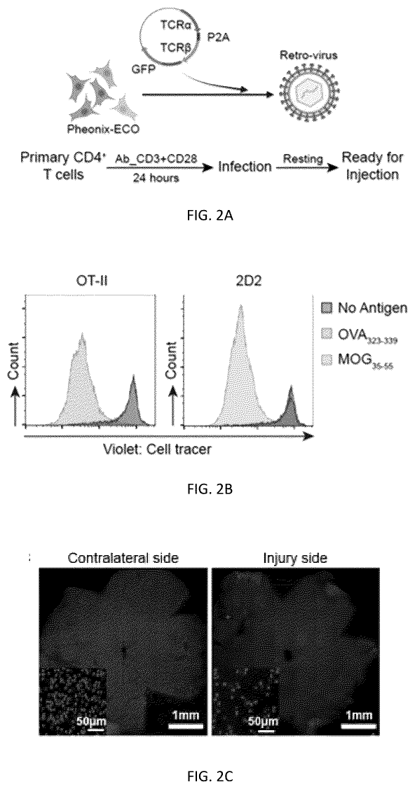

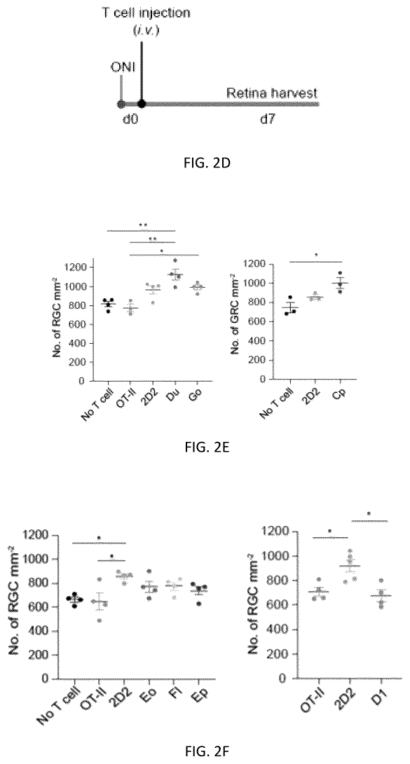

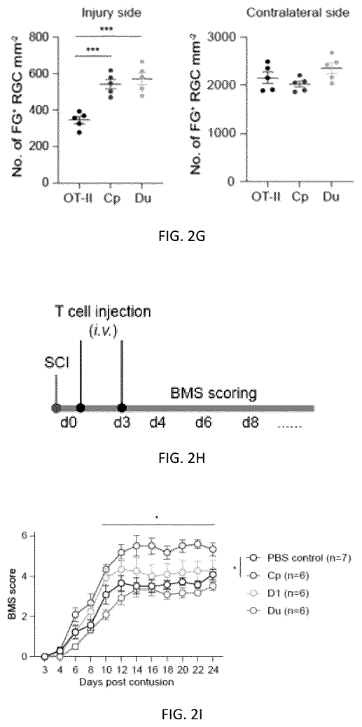

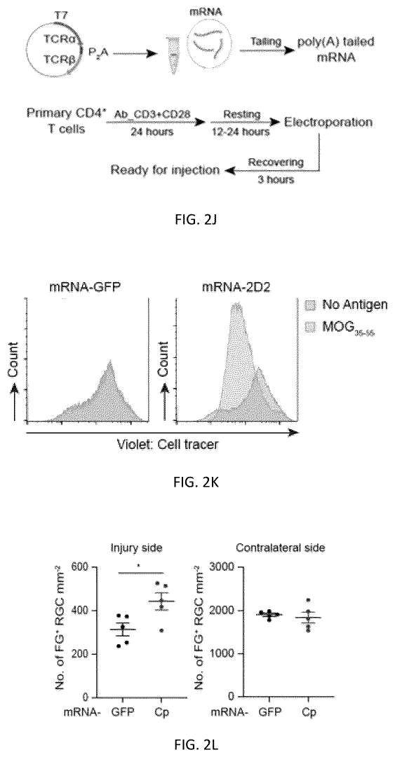

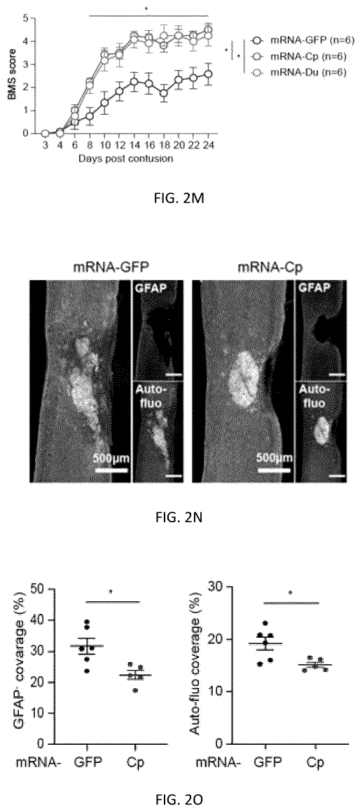

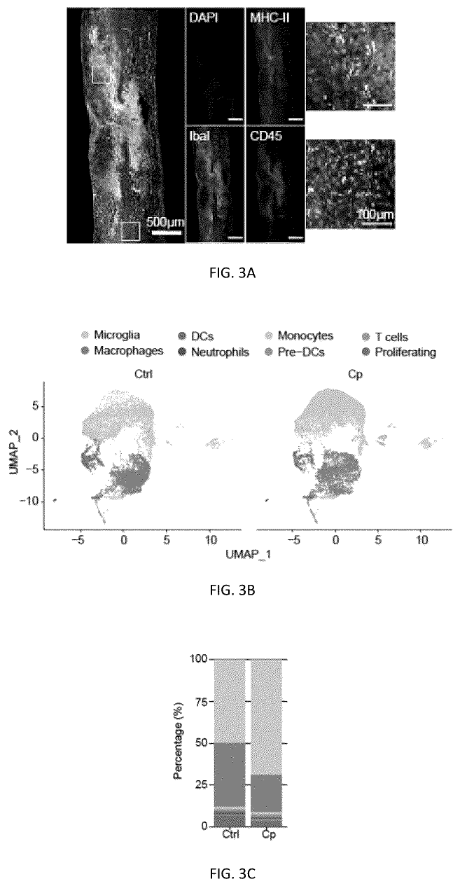

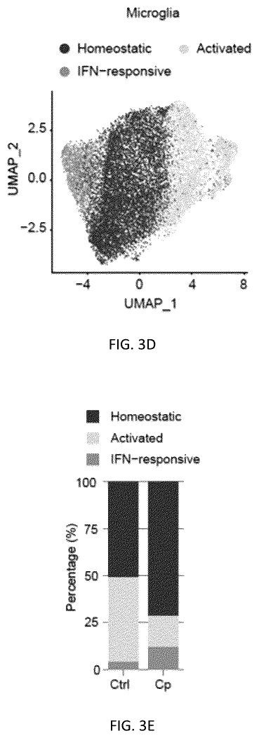

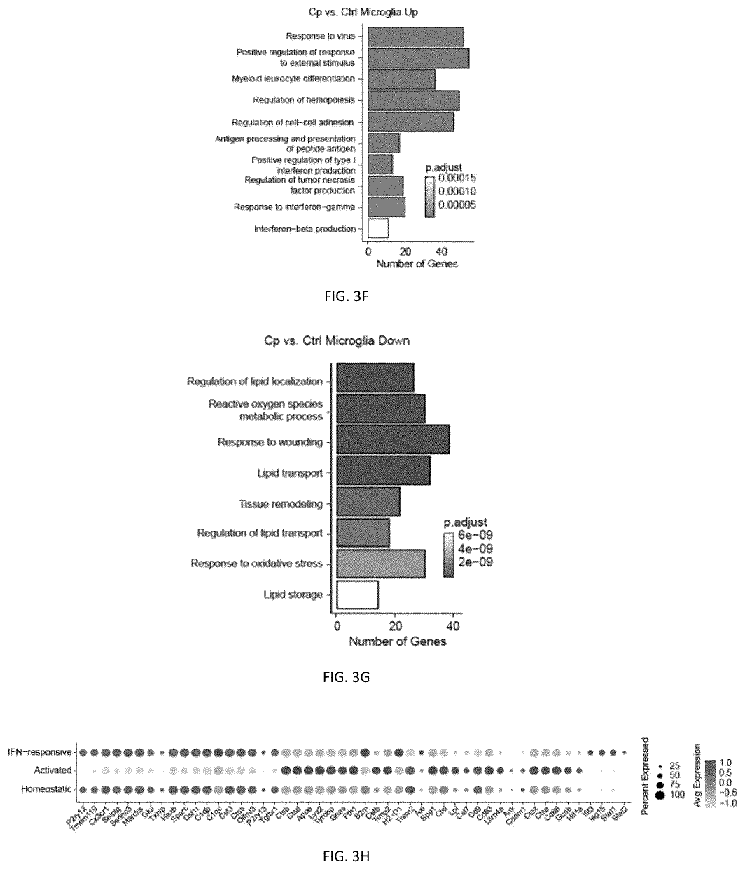

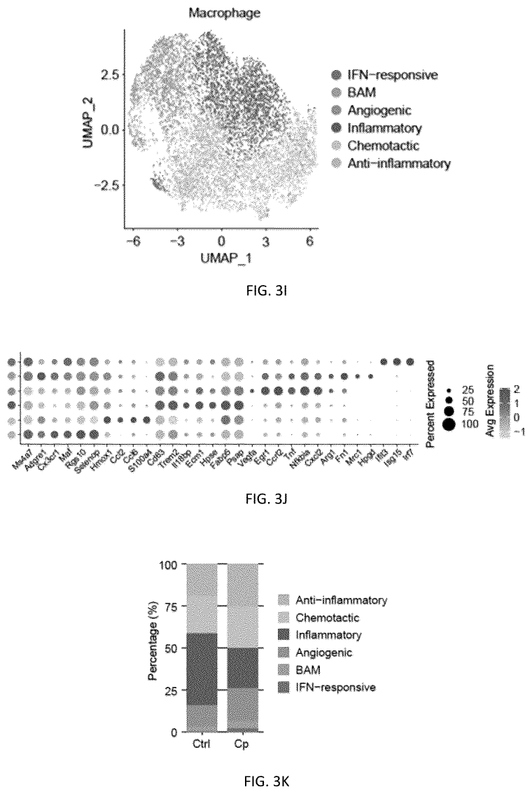

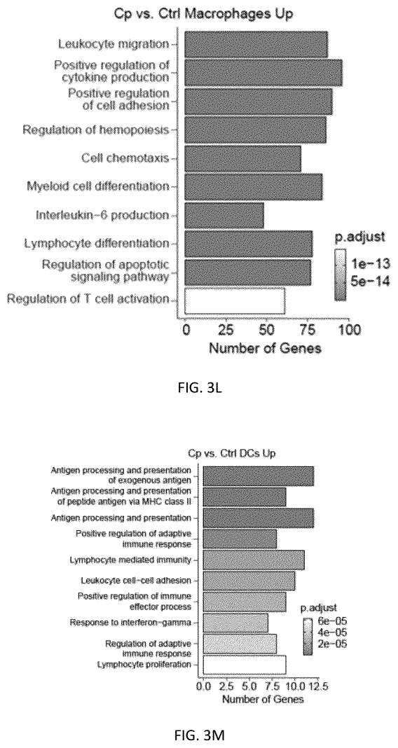

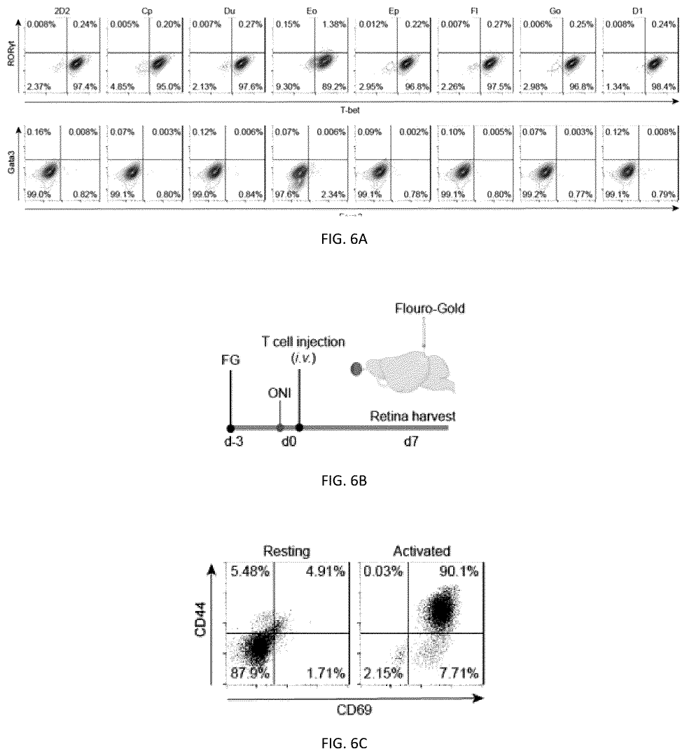

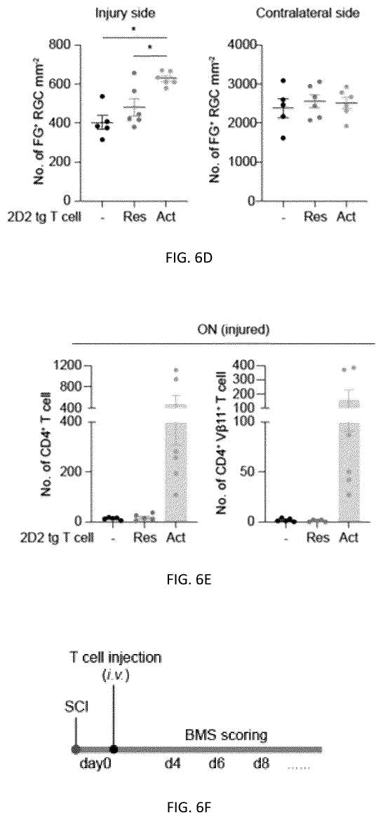

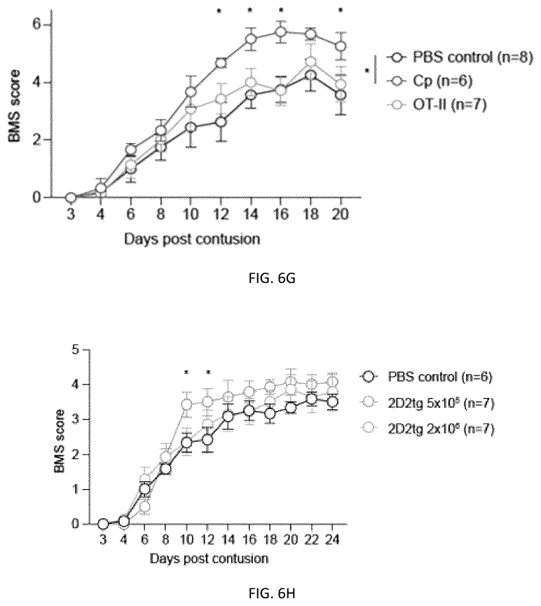

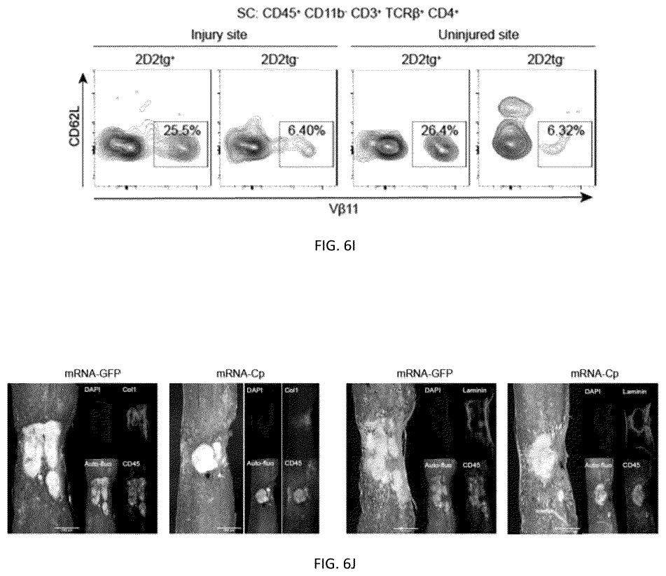

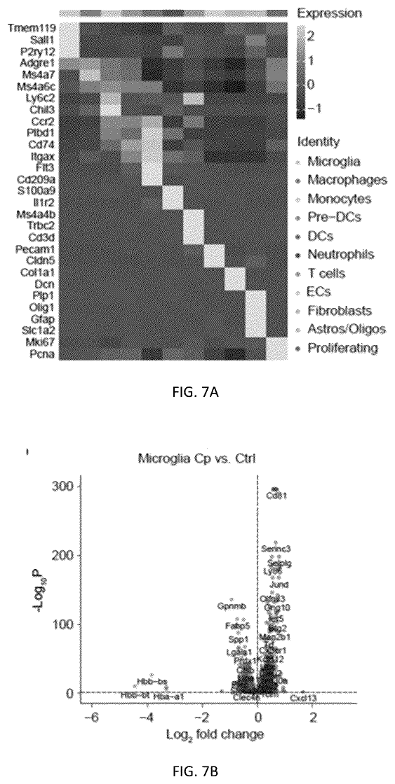

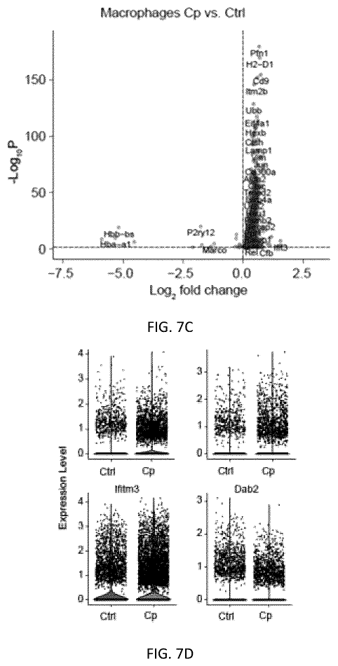

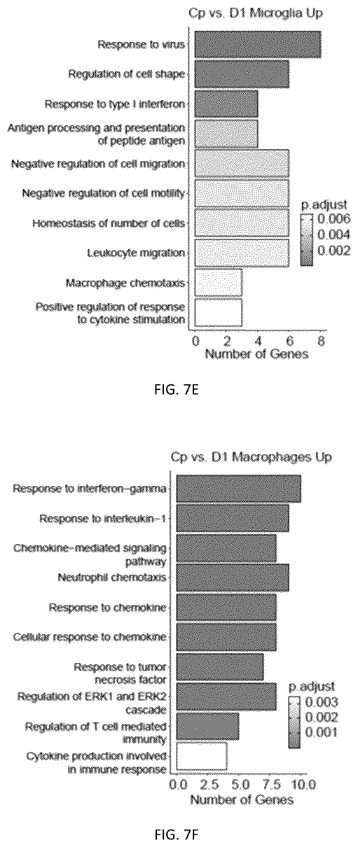

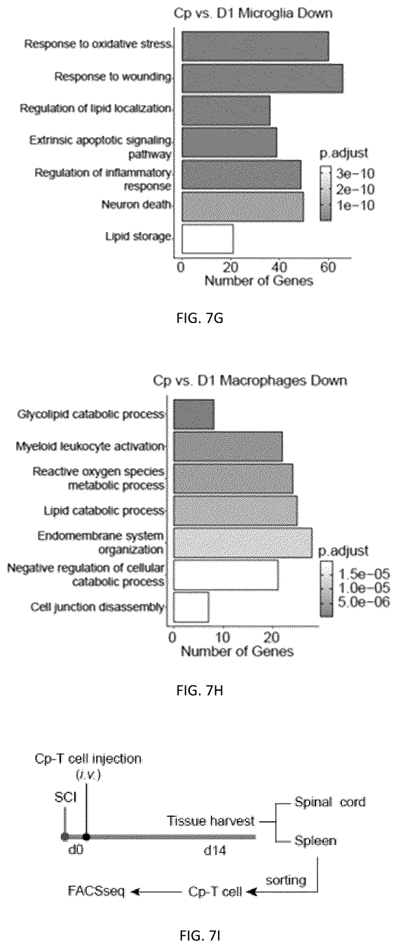

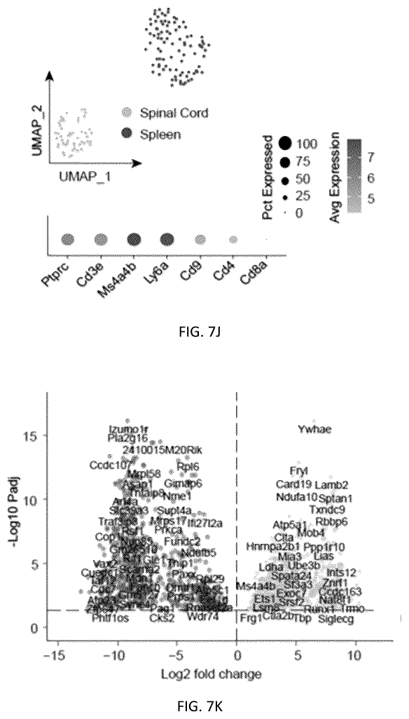

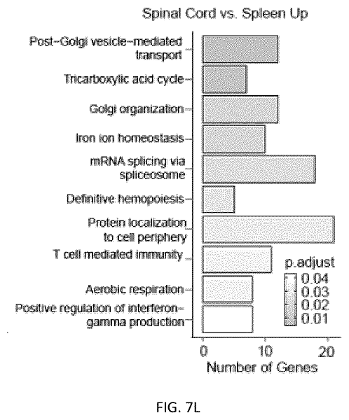

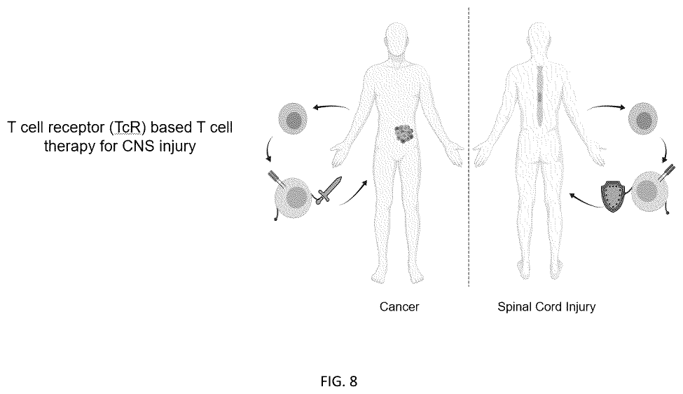

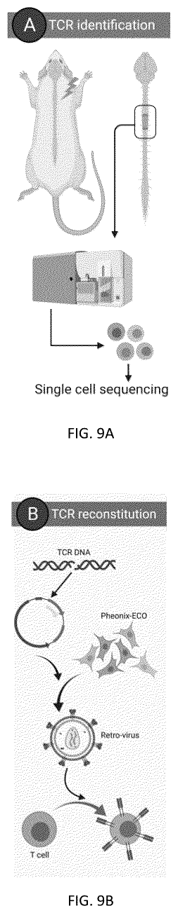

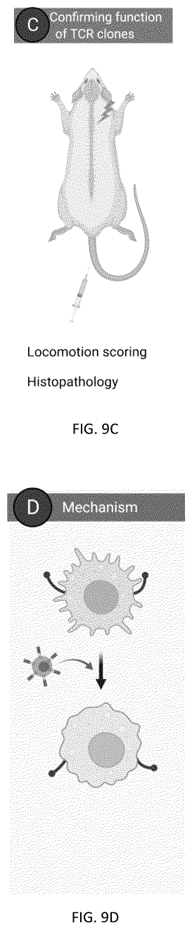

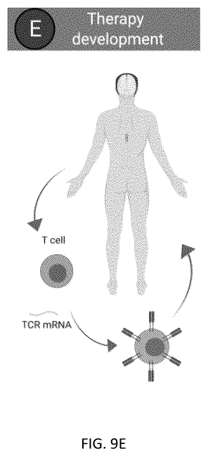

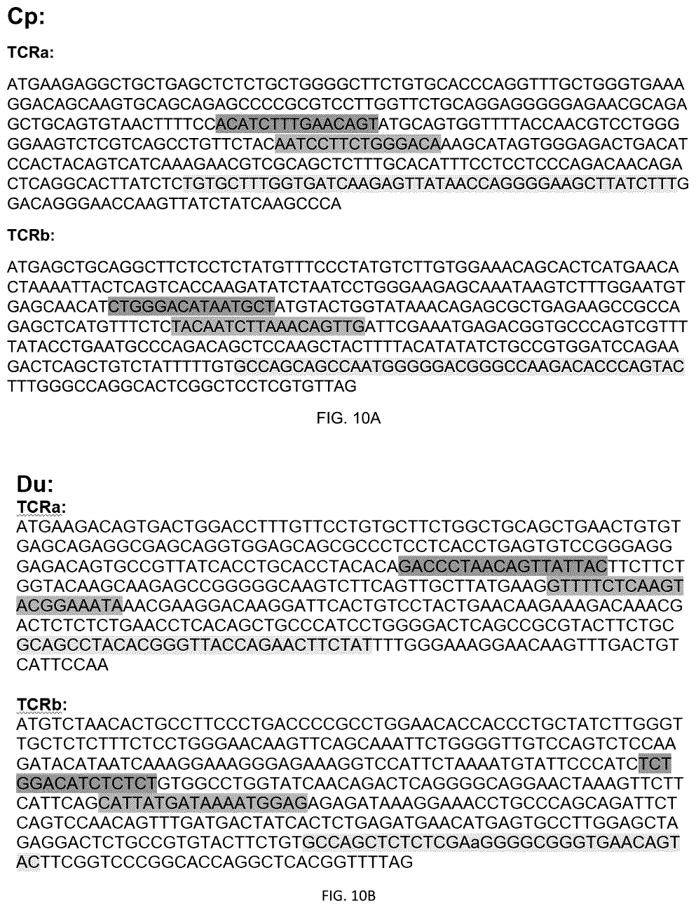

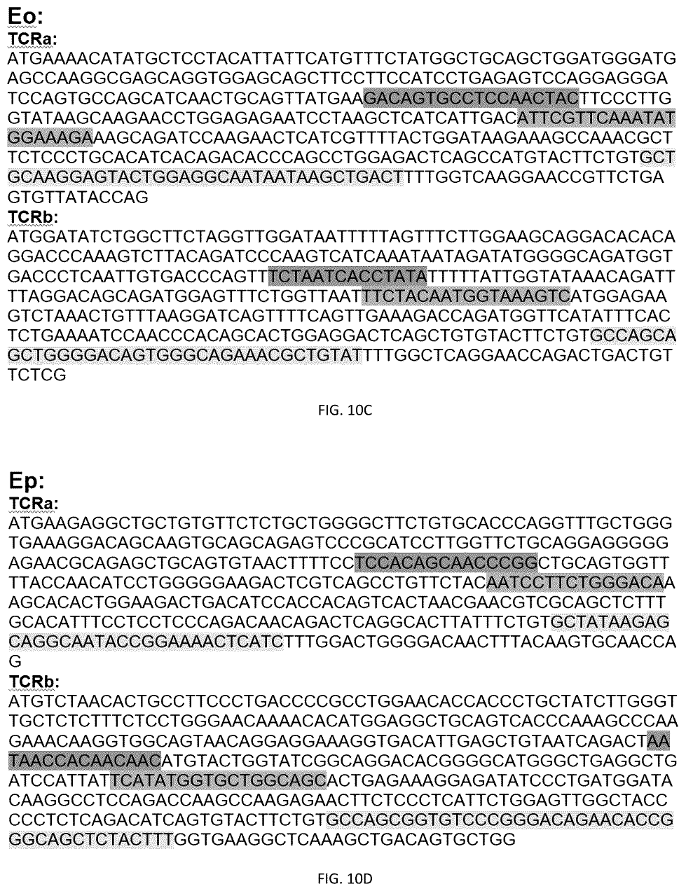

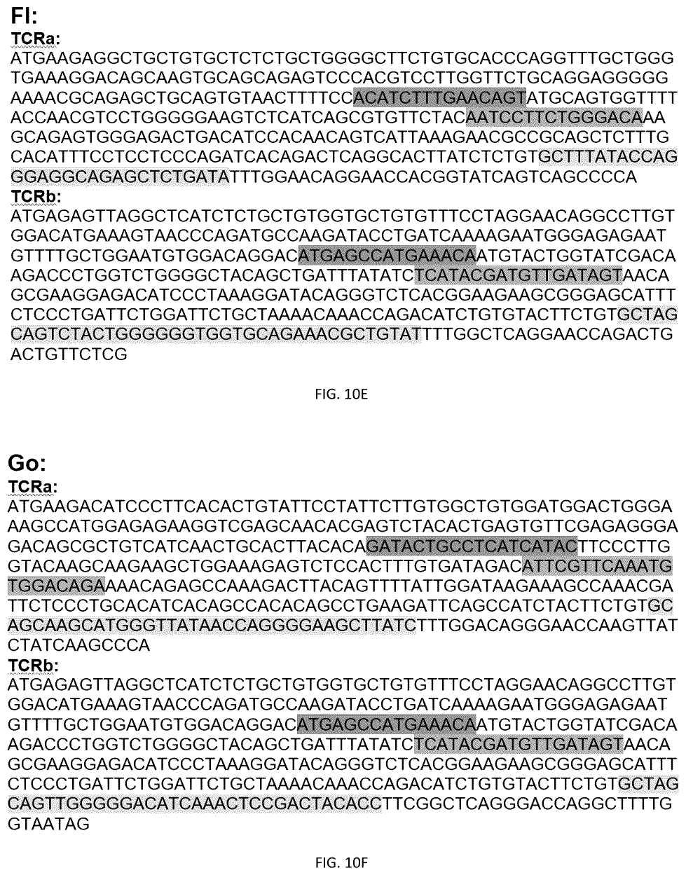

T cell therapy to treat CNS injury: The protective function of 2D2 TCR in optic nerve injury model leads to the examination of whether injury associated TCRs could be reconstituted to primary T cells and developed into a T cell therapy for CNS injury. A P2A link was used to link TCRα and TCRβ and retrovirus infection system used to reconstitute TCRs derived from V(D)J sequencing to infiltrating primary CD4⁺ T cells (FIG. 2A). OT-II and 2D2 TCR were reconstituted, the two TCRs well known recognizing OVA and MOG, in T cells separately and a T cell proliferation assay was used to test the function of reconstituted TCRs (FIG. 2B). Except clone Ei, which characterized as a Treg, all the CD4 clones were reconstituted, and were used in the optic nerve injury model as a functional screening assay to check the protective function of CNS-antigen specific TCR-expressing T cells. All the CD4⁺ T cells showed a Th1 feature in our system after expressing TCRs (FIG. 6A). Injury of optic nerve will eventually induce cell death of its retinal ganglion cell (RGC). Therefore, by counting number of alive RGCs (Brn-3a positive), a well quantified result representing the injury level of optic nerve survival is obtained (FIG. 2C). Surprisingly, compared with injury only or OT-II TCR group, all the spinal cord injury-derived TCR clones, either shows strong or weak, protective function for RGC recovery. The protective function of two clones, Cp and Du, was found to be even stronger than positive control 2D2 (FIG. 2D, FIG. 2E). Notably, clone Cp and Du were also shown to be the two clones with higher clonality relative to the others, demonstrating the association between antigen reactivity with neuron-protective function after CNS injury (FIG. 1I, FIG. 1J). the function of a TCR clone D1 was also tested from the diaphragm of naïve mice and found that this diaphragm clone doesn't have any protective function (FIG. 2F). Furthermore, Fluoro-Gold was injected into the SC region, which is the projection region of RGCs in brain, to allow the Fluoro-Gold to go through optic nerve and label RGCs. The protective effect observed for TCR Cp and Du in Fluoro-Gold injection assay was shown to be even stronger than that observed in the RGC staining assay (FIG. 2G). By comparing resting T cells with activated T cells, it was found that the activation of the T cell before injection is essential for the protective function (FIG. 6C, FIG. 6D, FIG. 6E).

After the best candidates were confirmed, Cp and Du, from the optic nerve injury screening, spinal cord injury was investigated to see if these two clones could behave similarly as in the optic nerve injury model. Different from what was observed in the optic nerve injury model, Cp works for both spinal cord injury model and optic nerve injury model and OT-II TCR or the diaphragm TCR did not have protective function. However, although Du worked well in optic nerve injury, it did not have any protective effect on spinal cord injury (FIG. 2H, FIG. 2I, FIG. 6F, FIG. 6G). This may be due to the different character of each tissue, however, it is possible that because spinal cord injury is a long-term model of injury and may allow effects of auto-immune T cells to be presented, whereas optic nerve injury, as a short-term model, would not be affected by auto-immune T cells. 2D2 T cells were titrated in spinal cord injury mice and results showed that high dose of 2D2 T cell did not have any protective effect and low dose of 2D2 T cell did have a protective effect in the early stage, but that the beneficial effect disappeared after day 10. T cell infiltration results also showed that after spinal cord injury, 2D2 T cells did not only infiltrate to injury site, but also to other part of the tissue (FIG. 6H and FIG. 6I). These results fit a model where auto-immune T cells benefit mouse recovery better from CNS injury in the early stage, but the beneficial effect is neutralized by adverse effect induced by potential auto-immune disease. Different from myelin sheet, neurons are usually wrapped by myelin sheet and the component in the neuron will not be exposed until the tissue is damaged. Thus, the reason why clone Cp, which recognize injury associated neuronal peptide Licam, doesn't have adverse effects may be because after tissue is healed, Cp will not meet its antigen anymore and therefore have no chance to induce auto-immune disease.

To minimize the side effect caused by auto-immune T cell and develop a better T cell therapy, a transient TCR expression system was generated using mRNA electroporation (FIG. 2J). The T cell proliferation assay was used to test the function of mRNA based TCR expression. In contrast with stably expression of TCRs, the mRNA system had a signal of cell tracer shift because these T cells just proliferate for limited generation (FIG. 2K). mRNA-TCR based T cell therapy also works for optic nerve injury. Different from stably expression of TCRs, by minimizing the side effect by transiently expressing TCRs, both Cp and Du worked well on spinal cord injury (FIG. 2M). The protective effect of T cell therapy is not only presented on locomotion, but also with the morphology of the scar. After treatment of therapeutic T cells, there is a smaller and more contained scar after mice are recovered from spinal cord injury (FIG. 2N, FIG. 2O). CD45 population in the scar and extracellular component like collagen and Laminin complement each other and fully fill the scar region (FIG. 6J).

What is claimed is:

1. A method of treating or preventing a central nervous system (CNS) injury in a subject in need thereof, the method comprising:
a) obtaining a biological sample from the subject at a CNS injury site wherein the sample comprises infiltrating lymphocytes;
b) isolating the infiltrating lymphocytes, and sequencing a T cell receptor (TCR) or part thereof, expressed by the lymphocytes;
c) isolating a MHC-peptide complex from the biological sample and identifying a pool of self-peptides;
d) expressing a T cell receptor identified in step b) in an immune effector cell;
e) screening a TCR-expressing immune effector cell generated in step d) against the self-peptide identified in step c) and selecting TCR-expressing immune effector cells with affinity to the self-peptide;
f) administering a population of TCR-expressing immune effector cells selected in step e) to the subject, thereby treating or preventing the CNS injury.

2. A method of treating or preventing neuronal death from a stroke a subject in need thereof, the method comprising:
a) obtaining a biological sample from the subject, wherein the sample comprises infiltrating lymphocytes;
b) isolating the infiltrating lymphocytes, and sequencing a T cell receptor (TCR) or part thereof expressed by the lymphocytes;
c) isolating a MHC-peptide complex from the biological sample identifying a pool of self-peptides;
d) expressing a T cell receptor identified in step b) in an immune effector cell;
e) screening a TCR-expressing immune effector cell generated in step d) against the self-peptide identified in step c) and selecting TCR-expressing immune effector cells with affinity to the self-peptide;
f) administering a population of TCR-expressing immune effector cells selected in step e) to the subject, thereby treating or preventing the CNS injury.

3. A method of treating or preventing neurodegeneration in a subject in need thereof, the method comprising:
a) obtaining a biological sample from the subject at a site of neurodegeneration, wherein the sample comprises infiltrating lymphocytes;
b) isolating the infiltrating lymphocytes, and sequencing a T cell receptor (TCR) or part thereof expressed by the lymphocytes;
c) isolating a MHC-peptide complex from the biological sample identifying a pool of self-peptides;
d) expressing a T cell receptor identified in step b) in an immune effector cell;
e) screening a TCR-expressing immune effector cell generated in step d) against the self-peptide identified in step c) and selecting TCR-expressing immune effector cells with affinity to the self-peptide;
f) administering a population of TCR-expressing immune effector cells selected in step e) to the subject, thereby treating or preventing the CNS injury.

4. The method of any one of claims 1 to 3, wherein the biological sample was previously obtained from the subject.

5. The method of any one of claims 1 to 4, wherein the biological sample is a tissue biopsy or cerebral spinal fluid.

6. The method of any one of claims 1 to 5, wherein the infiltrating lymphocytes comprise T cells.

7. The method of claim 6, wherein the T cells are CD4⁺ T cells or CD8⁺ T cells.

8. The method of any one of claims 1 to 7, wherein single-cell RNA sequencing is performed for single-cell assessment of cellular gene expression of the lymphocytes in step b).

9. The method of any one of claims 1 to 8, wherein the sequencing includes V(D)J sequencing.

10. The method of claim 8, wherein the cellular gene expression of the lymphocytes is compared to a reference gene expression from a T cell or population of T cells with naïve T cell features.

11. The method of claim 9, wherein lymphocytes are grouped by CDR3 region from the V(D)J sequencing and cells sharing the same sequence of TCRα and TCRβ pair are grouped as clones.

12. The method of any one of claims 1 to 11, wherein Mass Spec is used to identify the pool of self-peptides.

13. The method of claim 12, wherein the MHC is an MHC-II-peptide complex.

14. The method of any one of claims 1 to 13, wherein the identified TCR is stably expressed in the immune effector cell thereby generating the TCR-expressing immune effector cell

15. The method of any one of claims 1 to 13, wherein the identified TCR is expressed using mRNA thereby generating the TCR-expressing immune effector cell transiently.

16. The method of any one of claims 1 to 15, wherein the immune effector cell of step d) is a T cell.

17. The method of claim 16, wherein the T cell is a CD4⁺ T cell.

18. The method of any one of claims 1 to 17, wherein screening in step e) includes co-culturing TCR-expressing immune effector cells with a self-peptide identified and step c) and measuring proliferation and/or cytokine secretion.

19. The method of step 18, wherein ELISA or ELISpot is used.

20. The method of any one of claims 1 to 19, wherein the TCR-expressing immune effector cells which are activated by the self-peptides identified in step c) are selected for administration to the subject.

21. The method of claim 20, where in the cells are graded from low to high activation.

22. The method of claim 21, wherein the cells graded with low activation are selected for administration to the subject.

23. The method of any preceding claim, wherein the injured CNS tissue comprises an injured spinal cord, an injured brain, an injured retina, and any combination thereof.

24. The method of any preceding claim, wherein the CNS injury is associated with at least one of CNS trauma, autoimmunity, infection, aging, and chronic neurodegeneration.


SEQUENCE LISTING (32 sequences)

SEQ ID NO: 1 - protein, 12 aa, Mus musculus
CARSYNQGKLIF

SEQ ID NO: 2 - protein, 14 aa, Mus musculus
CASSLDPGANTEVF

SEQ ID NO: 3 - protein, 15 aa, Mus musculus
CALVIKSYNQGKLIF

SEQ ID NO: 4 - protein, 15 aa, Mus musculus
CASSQWGTGQDTQYF

SEQ ID NO: 5 - protein, 12 aa, Mus musculus
CAAYTGYQNFYF

SEQ ID NO: 6 - protein, 13 aa, Mus musculus
CASSLEGAGEQYF

SEQ ID NO: 7 - protein, 14 aa, Mus musculus
CAARSTGGNNKLTF

SEQ ID NO: 8 - protein, 14 aa, Mus musculus
CASSWGQWAETLYF

SEQ ID NO: 9 - protein, 13 aa, Mus musculus
CAIRAGNTGKLIF

SEQ ID NO: 10 - protein, 15 aa, Mus musculus
CASGVPGQNTGQLYF

SEQ ID NO: 11 - protein, 12 aa, Mus musculus
CALYQGGRALIF

SEQ ID NO: 12 - protein, 14 aa, Mus musculus
CASSLLGGAETLYF

SEQ ID NO: 13 - protein, 14 aa, Mus musculus
CAASMGYNQGKLIF

SEQ ID NO: 14 - protein, 14 aa, Mus musculus
CASSWGTSNSDYTF

SEQ ID NO: 15 - protein, 23 aa, Mus musculus
VTISSKIQKTKVEAPKLKVQHKF

SEQ ID NO: 16 - protein, 29 aa, Mus musculus
GPTRQASQAGPGPGPRTGPPTTQQPRPSG

SEQ ID NO: 17 - protein, 27 aa, Mus musculus
AGPGPRTGPPTTQQPRPSGPGPAGRPA

SEQ ID NO: 18 - protein, 20 aa, Mus musculus
DTGILDSIGRFFSGDRGAPK

SEQ ID NO: 19 - protein, 25 aa, Mus musculus
SHTRTTHYGSLPQKSQHGRTQDENP

SEQ ID NO: 20 - protein, 41 aa, Mus musculus
HGFLPRHRDTGILDSIGRFFSGDRGAPKRGSGKDSHTRTTH

SEQ ID NO: 21 - DNA, 394 bp, Mus musculus
atgaagaggctgctgagctctctgctggggcttctgtgcacccaggtttgctgggtgaaaggacagcaagtgcagcagagccccgcgtccttggttctgcaggagggggagaacgcagagctgcagtgtaacttttccacatctttgaacagtatgcagtggttttaccaacgtcctgggggaagtctcgtcagcctgttctacaatccttctgggacaaagcatagtgggagactgacatccactacagtcatcaaagaacgtcgcagctctttgcacatttcctcctcccagacaacagactcaggcacttatctctgtgctttggtgatcaagagttataaccaggggaagcttatctttggacagggaaccaagttatctatcaagccca

SEQ ID NO: 22 - DNA, 397 bp, Mus musculus
atgagctgcaggcttctcctctatgtttccctatgtcttgtggaaacagcactcatgaacactaaaattactcagtcaccaagatatctaatcctgggaagagcaaataagtctttggaatgtgagcaacatctgggacataatgctatgtactggtataaacagagcgctgagaagccgccagagctcatgtttctctacaatcttaaacagttgattcgaaatgagacggtgcccagtcgttttatacctgaatgcccagacagctccaagctacttttacatatatctgccgtggatccagaagactcagctgtctatttttgtgccagcagccaatgggggacgggccaagacacccagtactttgggccaggcactcggctcctcgtgttag

SEQ ID NO: 23 - DNA, 397 bp, Mus musculus
atgaagacagtgactggacctttgttcctgtgcttctggctgcagctgaactgtgtgagcagaggcgagcaggtggagcagcgccctcctcacctgagtgtccgggagggagacagtgccgttatcacctgcacctacacagaccctaacagttattacttcttctggtacaagcaagagccgggggcaagtcttcagttgcttatgaaggttttctcaagtacggaaataaacgaaggacaaggattcactgtcctactgaacaagaaagacaaacgactctctctgaacctcacagctgcccatcctggggactcagccgcgtacttctgcgcagcctacacgggttaccagaacttctattttgggaaaggaacaagtttgactgtcattccaa

SEQ ID NO: 24 - DNA, 424 bp, Mus musculus
atgtctaacactgccttccctgaccccgcctggaacaccaccctgctatcttgggttgctctctttctcctgggaacaagttcagcaaattctggggttgtccagtctccaagatacataatcaaaggaaagggagaaaggtccattctaaaatgtattcccatctctggacatctctctgtggcctggtatcaacagactcaggggcaggaactaaagttcttcattcagcattatgataaaatggagagagataaaggaaacctgcccagcagattctcagtccaacagtttgatgactatcactctgagatgaacatgagtgccttggagctagaggactctgccgtgtacttctgtgccagctctctcgaaggggcgggtgaacagtacttcggtcccggcaccaggctcacggttttag

SEQ ID NO: 25 - DNA, 403 bp, Mus musculus
atgaaaacatatgctcctacattattcatgtttctatggctgcagctggatgggatgagccaaggcgagcaggtggagcagcttccttccatcctgagagtccaggagggatccagtgccagcatcaactgcagttatgaagacagtgcctccaactacttcccttggtataagcaagaacctggagagaatcctaagctcatcattgacattcgttcaaatatggaaagaaagcagatccaagaactcatcgttttactggataagaaagccaaacgcttctccctgcacatcacagacacccagcctggagactcagccatgtacttctgtgctgcaaggagtactggaggcaataataagctgacttttggtcaaggaaccgttctgagtgttataccag

SEQ ID NO: 26 - DNA, 400 bp, Mus musculus
atggatatctggcttctaggttggataatttttagtttcttggaagcaggacacacaggacccaaagtcttacagatcccaagtcatcaaataatagatatggggcagatggtgaccctcaattgtgacccagtttctaatcacctatatttttattggtataaacagattttaggacagcagatggagtttctggttaatttctacaatggtaaagtcatggagaagtctaaactgtttaaggatcagttttcagttgaaagaccagatggttcatatttcactctgaaaatccaacccacagcactggaggactcagctgtgtacttctgtgccagcagctggggacagtgggcagaaacgctgtattttggctcaggaaccagactgactgttctcg

SEQ ID NO: 27 - DNA, 388 bp, Mus musculus
atgaagaggctgctgtgttctctgctggggcttctgtgcacccaggtttgctgggtgaaaggacagcaagtgcagcagagtcccgcatccttggttctgcaggagggggagaacgcagagctgcagtgtaacttttcctccacagcaacccggctgcagtggttttaccaacatcctgggggaagactcgtcagcctgttctacaatccttctgggacaaagcacactggaagactgacatccaccacagtcactaacgaacgtcgcagctctttgcacatttcctcctcccagacaacagactcaggcacttatttctgtgctataagagcaggcaataccggaaaactcatctttggactggggacaactttacaagtgcaaccag

SEQ ID NO: 28 - DNA, 430 bp, Mus musculus
atgtctaacactgccttccctgaccccgcctggaacaccaccctgctatcttgggttgctctctttctcctgggaacaaaacacatggaggctgcagtcacccaaagcccaagaaacaaggtggcagtaacaggaggaaaggtgacattgagctgtaatcagactaataaccacaacaacatgtactggtatcggcaggacacggggcatgggctgaggctgatccattattcatatggtgctggcagcactgagaaaggagatatccctgatggatacaaggcctccagaccaagccaagagaacttctccctcattctggagttggctaccccctctcagacatcagtgtacttctgtgccagcggtgtcccgggacagaacaccgggcagctctactttggtgaaggctcaaagctgacagtgctgg

SEQ ID NO: 29 - DNA, 385 bp, Mus musculus
atgaagaggctgctgtgctctctgctggggcttctgtgcacccaggtttgctgggtgaaaggacagcaagtgcagcagagtcccacgtccttggttctgcaggagggggaaaacgcagagctgcagtgtaacttttccacatctttgaacagtatgcagtggttttaccaacgtcctgggggaagtctcatcagcgtgttctacaatccttctgggacaaagcagagtgggagactgacatccacaacagtcattaaagaacgccgcagctctttgcacatttcctcctcccagatcacagactcaggcacttatctctgtgctttataccagggaggcagagctctgatatttggaacaggaaccacggtatcagtcagcccca

SEQ ID NO: 30 - DNA, 400 bp, Mus musculus
atgagagttaggctcatctctgctgtggtgctgtgtttcctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagtctactggggggtggtgcagaaacgctgtattttggctcaggaaccagactgactgttctcg

SEQ ID NO: 31 - DNA, 403 bp, Mus musculus
atgaagacatcccttcacactgtattcctattcttgtggctgtggatggactgggaaagccatggagagaaggtcgagcaacacgagtctacactgagtgttcgagagggagacagcgctgtcatcaactgcacttacacagatactgcctcatcatacttcccttggtacaagcaagaagctggaaagagtctccactttgtgatagacattcgttcaaatgtggacagaaaacagagccaaagacttacagttttattggataagaaagccaaacgattctccctgcacatcacagccacacagcctgaagattcagccatctacttctgtgcagcaagcatgggttataaccaggggaagcttatctttggacagggaaccaagttatctatcaagccca

SEQ ID NO: 32 - DNA, 397 bp, Mus musculus
atgagagttaggctcatctctgctgtggtgctgtgtttcctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttgggggacatcaaactccgactacaccttcggctcagggaccaggcttttggtaatag